(19) United States
(12) Patent Application Publication
(10) Pub. No.: US 2020/0306183 A1
(43) Pub. Date: Oct. 1, 2020

(54) POLYMER COMPOSITE FOR CONTROLLED RELEASE OF AN AGENT

(71) Applicant: Commonwealth Scientific and Industrial Research Organisation, Acton (AU)

(72) Inventors: Timothy Hughes, Clayton, Victoria (AU); Xavier Mulet, Clayton, Victoria (AU); Johan Basuki, Clayton, Victoria (AU); Xiaojuan Hao, Clayton, Victoria (AU)

(73) Assignee: Commonwealth Scientific and Industrial Research Organisation, Acton (AU)

(21) Appl. No.: 16/061,772

(22) PCT Filed: Dec. 16, 2016
(86) PCT No.: PCT/AU2016/051249

§ 371 Date: Jun. 13, 2018

(30) Foreign Application Priority Data
Dec. 18, 2015 (AU) 2015905261

(51) Int. Cl. A61K 9/00; A61K 47/34; A61K 47/36; A61K 47/02

(52) U.S. Cl. CPC A61K 9/0051; A61K 47/34; A61K 9/70; A61K 47/02; A61K 47/36

(57) ABSTRACT

The present invention relates to a polymer composite for controlled release of an agent. In particular, the present invention relates to a stimuli-responsive polymer composite that can provide for controlled release of an agent in response to light. The polymer composite may be particularly suitable for the controlled delivery of a drug that is modulated by light.

### TECHNICAL FIELD

The present invention relates to a polymer composite for controlled release of an agent. In particular, the present invention relates to a polymer composite that can be stimulated by light to provide for controlled release of an agent.

### BACKGROUND

Smart polymer systems that can be triggered to release an agent are of considerable interest for a range of applications. In particular, such polymer systems are of interest for drug delivery applications, where it can be desirable to provide for release of a drug under specified conditions or at a specified time.

Polymer systems capable of providing triggered release of an agent may have a stimuli responsive polymer component. The stimuli responsive polymer can undergo a reversible chemical, physical or solubility change in response to an external stimulus, such as temperature, pH, enzymes, microwave radiation, magnetic fields or light. The stimulus can be applied to the polymer material in order to induce a change in the material, which allows the agent to be released.

Light is viewed as an attractive stimulus as characteristics such as light intensity, wavelength and illumination time can be easily adjusted and controlled. Polymer composites that respond to light may be capable of undergoing an immediate change upon light exposure, enabling an agent contained in the composite to be released on demand at a particular time or at a particular site. Furthermore, the amount of agent released or its rate of release may be modulated by adjusting the duration of light exposure or the wavelength and/or intensity of the light.

Polymer composites that are capable of being stimulated by light can broadly fall into two categories, which can be classified by reference to the mechanism by which the polymer composite responds to light in order to release an encapsulated payload.

One type of light responsive polymer composite utilises chemical linkages in the polymer composite that are cleavable when the composite is exposed to light. Cleavage of the chemical linkages result in breakdown of the composite, which allows an agent contained in the composite to be released. However, a problem with the polymer composite is that the mechanism governing the release of the agent is irreversible due to permanent cleavage of the linkages. Moreover, cleavage of the linkages and release of the agent can be difficult to control, which might lead to burst release of the agent.

Another type of light responsive polymer composite utilises inorganic photothermal particles dispersed in a synthetic crosslinked poly(N-isopropylacrylamide) (PNIPAAm) hydrogel matrix, which has been envisaged for use in drug delivery applications. In such composites, thermal energy generated by the photothermal particles upon exposure of the composite to light is used to heat the PNIPAAm above its lower critical solution temperature (LCST). Upon heating above the LCST, PNIPAAm becomes hydrophobic. This change causes the polymer to shrink or collapse in the aqueous environment, resulting in an encapsulated drug being squeezed out from the polymer. However, one issue with composites formed with synthetic polymers is that they can contain residual unreacted monomers, which can be toxic to biological systems. As a result, multiple purification steps may need to be carried out during preparation of the composites in order to improve their biocompatibility.

Another present shortcoming with light responsive polymer composites is that direct drug loading in the crosslinked polymer of the composite may be limited by the amount of drug that can be absorbed in the polymer and the molecular size of the drug that can be loaded into a pre-formed composite. While drug loading might be improved by fabricating the composite in the form of a membrane containing a drug reservoir that is separated from a thermoplastic polymer matrix, there can be limited anisotropic light control over drug release from the reservoir. Fabrication of the light responsive polymer composite into a membrane may also involve complex procedures and may limit the versatility of the composite for the delivery of different classes of drugs. Furthermore, light responsive polymer composite membranes may only be administered to certain body sites of a patient since administration would generally involve a surgical procedure, thus limiting the range of drug delivery applications.

It would be desirable to provide a polymer composite that addresses or at least ameliorates one or more disadvantages of existing polymer composites and which can be triggered by light to provide for controlled release of an agent. In particular, it would be desirable to provide a light responsive polymer composite that provides greater control over drug delivery.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date of each claim of this application.

### SUMMARY OF THE INVENTION

The present invention relates generally to polymer composites that are responsive to light. The polymer composites contain an agent, with release of the agent from the composite being modulated by light.

In one aspect, the present invention provides a polymer composite for controlled release of an agent comprising:
a thermoplastic polymer matrix;
an agent dispersed in the thermoplastic polymer matrix; and
a plurality of photo-thermal particles dispersed in the thermoplastic polymer matrix, the particles having a non-carboxylic acid stabiliser bound thereto,

wherein the thermoplastic polymer matrix has a softening point when in the polymer composite at a temperature in the range of from about 30° C. to 70° C. as determined by rheometer, and wherein upon exposure of the composite to photo energy, the particles absorb the photo energy and emit thermal energy to promote the softening of the thermoplastic polymer matrix and modulate the release of the agent from the thermoplastic polymer matrix.

The softening point of the polymer matrix can be determined by dynamic mechanical analysis (DMA) using an oscillatory shear rheometer. Using such techniques, softening of the thermoplastic polymer matrix in the composite is indicated by a decrease in the storage modulus (E′ or G′) and loss modulus (E″ and G″) of the polymer matrix with an increase in temperature.

As explained further below, the softening point of the thermoplastic polymer matrix is characterised by a decrease in storage modulus as determined by rheometer, which occurs the temperature range of from about 30° C. to 70° C. The softening point may also be referred to herein as a softening temperature.

A skilled person would appreciate that changes in modulus can be directly correlated with changes in the viscosity of the thermoplastic polymer matrix as determined by rheometer. In particular, viscosity changes are directly proportional to modulus changes, such that viscosity also decreases with an increase in temperature. Thermoplastic polymer matrices having a softening point in the temperature range of from about 30° C. to 70° C. can also exhibit a decrease in viscosity in this temperature range, as measured by rheometer.

The softening point can be determined in accordance with procedures described in accepted international standards. In one embodiment, the softening point is determined in accordance with ASTM-E1640.

The polymer composite of the invention comprises a plurality of photo-thermal particles that convert photo-energy into thermal energy. The thermal energy heats the thermoplastic polymer matrix up to at least the softening point, thus leading to softening of the thermoplastic polymer matrix and the release of the agent contained in the matrix to the surrounding environment. By “softening” of the thermoplastic polymer matrix is meant that the thermoplastic polymer matrix in the composite becomes more pliable or malleable when the composite is heated in the desired temperature range. The onset of softening of the thermoplastic polymer matrix occurs once the softening point of the matrix has been reached.

One feature of the invention is that the photo-thermal particles have non-carboxylic acid stabiliser bound to them. The stabiliser may be a non-carboxylic acid polymeric stabiliser or a non-carboxylic acid oligomeric stabiliser. The stabiliser helps to ensure that the photo-thermal particles are more homogenously dispersed in the thermoplastic polymer matrix. A more homogeneous distribution enables more even heating of the thermoplastic polymer matrix to be achieved.

In some embodiments of the invention, the non-carboxylic acid stabiliser comprises a non-carboxylic acid polymer. In such embodiments, the stabiliser may be a non-carboxylic acid polymeric stabiliser comprising a neutral (uncharged) or charged polymer. A polymeric stabiliser may help to stabilise the photo-thermal particles in the thermoplastic polymer matrix via electrostatic effects, steric effects, hydrogen bonding interactions, hydrophobic interactions, or any combination thereof.

In some embodiments, the non-carboxylic stabiliser comprises a charged polymer, preferably a cationic polymer. In an exemplary embodiment, the polymeric stabiliser comprises poly(methacryloyloxyethyl trimethyl ammonium chloride).

In other embodiments, the non-carboxylic stabiliser comprises a neutral polymer. In an exemplary embodiment, the polymeric stabiliser comprises poly(oligoethylene glycol methacrylate).

In some embodiments of the invention, the non-carboxylic acid stabiliser is a non-carboxylic acid oligomeric stabiliser.

The non-carboxylic acid stabiliser may be bound to the photo-thermal particles by covalent or non-covalent interactions. Preferably, the stabiliser is covalently bound to the surface of the photo-thermal particles. In exemplary embodiments, the non-carboxylic acid stabiliser is covalently bound to the photo-thermal particles via a functional group selected from the group consisting of a thiol, thiocarbonylthio and amino functional group, preferably a thiol functional group.

The thermoplastic polymer matrix softens in the polymer composite in response to thermal energy or heat emitted by the photo-thermal particles. Softening of the thermoplastic polymer matrix results in a modulation of the release of the agent contained therein. In some embodiments, the thermoplastic polymer matrix has a softening point when in the composite at a temperature in a range selected from the group consisting of from 35° C. to 65° C., from 37° C. to 60° C., and from 40° C. to 55° C. The temperature at which the thermoplastic polymer matrix softens (i.e. its softening temperature or softening point) is determined by rheometer.

In another set of embodiments the thermoplastic polymer matrix is in the form of a hydrogel, which comprises a polymer phase and an aqueous liquid phase. The polymer phase may form between 0.01 to 90% (w/w) of the hydrogel, depending on the type of polymer used to form the polymer phase.

Some exemplary hydrogels have a polymer phase comprising a polymer selected from the group consisting of a polysaccharide, a polypeptide, a polyether, a polyester, a poly(vinyl alcohol), a poly(vinyl pyrrolidone), a poloxamer and combinations thereof.

In one set of embodiments, the polymer phase of the hydrogel comprises a polysaccharide selected from the group consisting of agarose, carrageenan, chitosan, gellan gum, alginate, hyaluronic acid, cellulose, starch, and mixtures thereof, preferably agarose.

In another set of embodiments, the hydrogel comprises a polypeptide selected from the group consisting of collagen and gelatine, preferably gelatine.

In another set of embodiments, the hydrogel comprises a synthetic hydrophilic polymer selected from the group consisting of polyether, poloxamer (Pluronic®), polyester, poly(vinyl pyrrolidone) poly(ethylene-vinyl acetate) and poly(vinyl alcohol), preferably poloxamer. Exemplary poloxamers may be selected from the group consisting of poloxamer 407 (also known as Pluronic® F127), poloxamer 338 (also known as Pluronic® F108), and poloxamer 237 (also known as Pluronic® F87).

The polymer phase may form between 0.01 to 90% (w/w) of the hydrogel, although this may depend on the polymer present in the hydrogel.

In some embodiments, the thermoplastic polymer matrix is an agarose hydrogel comprising from 0.01 to 10%, from 0.03 to 5%, from 0.05 to 3%, or from 0.1 to 1% of agarose as the polymer phase.

In some embodiments, the thermoplastic polymer matrix is a gelatine hydrogel comprising from 0.1 to 10% gelatin as the polymer phase.

In some embodiments, the thermoplastic polymer matrix is a poloxamer hydrogel comprising from 5 to 50%, from 10 to 40% or from 20 to 30% of a poloxamer as the polymer phase.

In one set of embodiments, the thermoplastic polymer matrix is in a neat (or dry) form. This means that the thermoplastic polymer matrix is generally not hydrated or solvated by a solvent.

The thermoplastic polymer matrix may be in the form of a neat polymer film.

In some embodiments, the thermoplastic polymer matrix may comprise a neat polymer selected from the group consisting of polyesters (e.g. polycaprolactone), polyamides (e.g. nylon 6), polyoxazolines, polyethers (e.g. poly(ethylene glycol)), polyvinyl polymers (e.g. poly(vinyl pyrrolidone), poly(ethylene-vinyl acetate) and poly(vinyl alcohol)), and combinations thereof. These polymer matrices have softening points in the temperature range of 30° C. to 70° C.

In one set of embodiments, the thermoplastic polymer matrix of the polymer composite comprises a neat polyester. The neat polyester may be a homopolymer or copolymer of at least one monomer selected from the group consisting of ε-caprolactone, lactic acid, glycolic acid, lactide and glycolide. In one form, the thermoplastic polymer matrix comprises neat polycaprolactone. Polycaprolactone in the thermoplastic polymer matrix of the composite may have a molecular weight (Mₙ) in a range of from about 1000 g/mol to 43,000 g/mol.

The photo-thermal particles present in the polymer composite can absorb photo-energy of one or more wavelengths. In some embodiments, polymer composites according to the invention comprise photo-thermal particles that absorb photo-energy having a wavelength in a range selected from the group consisting of from about 10 nm to about 1 mm, from about 365 nm to about 1400 nm, and from about 400 nm to about 900 nm.

A variety of photo-thermal particles may be suitably employed. In one set of embodiments, photo-thermal particles present in the polymer composite may be metallic particles.

In a particular set of embodiments, the photo-thermal particles are gold particles. Gold particles useful as photo-thermal particles may have a diameter in a range selected from the group consisting of from about 5-400 nm, from about 10-200 nm, from about 20-100 nm, and from about 40-80 nm. The gold particles may be present in the polymer composite in an amount of from about 0.01 to 10.0 mg/ml, or 0.001 to 1.0% (w/v).

The polymer composite of the invention also comprises an agent that is capable of being released from the thermoplastic polymer matrix. In one form, the agent contained in the thermoplastic polymer matrix of the polymer composite may be a drug. In some embodiments, the drug is selected from the group consisting of a therapeutic agent, a diagnostic agent, a prophylactic agent, and combinations thereof. In particular embodiments, the drug is selected from the group consisting of biologically active macromolecules, small molecules, organometallic compounds, nucleic acids, isotopically labeled chemical compounds, and combinations thereof.

In a particular set of embodiments, the polymer composite may be injectable and is formed into a shape such that that it can pass through the lumen of a needle for administration by injection to a desired site. The polymer composite may be in the form of particles (such as spherical particles) or cylindrical rods. Particles or rods formed from the composite may have at least one dimension (e.g. a diameter) in the range of from 1 μm to 1000 μm. In some embodiments, particles or rods preferably have a diameter in a range of from 10 to 200 μm. Polymer composites in the form of microparticles can be preferred if the composite is to act as a drug depot for local drug delivery. Furthermore, unlike nanoparticles, microparticles are not taken up by cells and migration of the microparticles into blood and lymph vessels can be minimised.

In one form, the polymer composite is in the form of particles, preferably spherical particles, more preferably microparticles, each particle being contained in an additional thermoplastic polymer, preferably a thermoplastic hydrogel.

In another aspect the present invention provides a process for the preparation of a polymer composite of one or more embodiments described herein, the process comprising the steps of forming a liquid polymer mixture comprising at least one polymer, at least one agent and a plurality of non-carboxylic acid stabilised photo-thermal particles, and solidifying the liquid polymer mixture to form the polymer composite.

The polymer contained in the liquid polymer mixture forms part of the thermoplastic polymer matrix of the polymer composite.

In one embodiment, solidification of the liquid polymer mixture may involve the step of crosslinking the polymer contained in the polymer mixture.

Solidification of the liquid polymer mixture may be achieved by cooling the liquid polymer mixture to a desired temperature. In one preference, the liquid polymer mixture is cooled to a temperature that is less then 30° C.

In other embodiments, solidification of the liquid polymer mixture may be achieved by heating the liquid polymer mixture to a desired temperature. In one preference, the liquid polymer mixture is heated to a temperature that is greater then 30° C.

In one embodiment, solidification of the liquid polymer mixture involves dispersion of the liquid mixture in a continuous phase to form a plurality of discrete polymer composite particles, preferably polymer composite microparticles. In some embodiments the liquid polymer mixture is dispersed in the continuous phase dropwise or under shear.

In another aspect, the present invention provides an implantable article comprising a polymer composite of any of the embodiments described herein contained within a thermoplastic polymer. In the implantable article, the polymer composite may be coated or enclosed by the thermoplastic polymer. Preferably, the implantable article is injectable.

The polymer composite of the invention may suitably be used for drug delivery applications, where controlled release of drug is desired. In particular, the polymer composite of the invention may be formed into or contained in a drug delivery article or device that may be implanted at a desired body site.

In one set of embodiments, the polymer composite is used for ocular drug delivery for the administration of a drug to an eye of a subject. In some embodiments, ocular drug delivery may involve the implantation of a device or article comprising a polymer composite of the invention in at least one eye of a subject. In such embodiments, the polymer composite may be prepared in a form that is suitable for implantation into the eye.

In another aspect, the present invention therefore also provides an ocular implant comprising a polymer composite of any one of the embodiments described herein.

In another set of embodiments, the polymer composite is used for subcutaneous drug delivery. The polymer composite may be incorporated in an article or device that is fabricated for subcuteaneous implantation.

In another aspect, the present invention therefore also provides a subcutaneous implant comprising a polymer composite of any one of the embodiments described herein.

The polymer composite of the invention may provide a reservoir of an agent, which can be released on demand upon exposure of the composite to light. This can be particularly advantageous for implants, which can therefore provide a drug reservoir, with release of a payload of the drug taking place when desired by exposing the implant to light.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

### BRIEF DESCRIPTION OF THE FIGURES

Embodiments of the invention will now be described with reference to the following non-limiting drawings in which:

FIG. 1 is a scheme illustrating embodiments of polymer composites of the invention fabricated with a neat polymer or hydrogel thermoplastic polymer matrix, which can be modified to be in the form of spherical particles and optionally coated or enclosed within another thermoplastic polymer.

FIG. 2 is graph showing the light-modulated temperature increase and release profile of lysozyme (2.5 mg·ml⁻¹ [lysozyme] in 4% agarose+0.3 mg·ml⁻¹ AuNPs) over period of time, where the “ON” stage indicates the exposure of blue light to the AuNPs/hydrogel composite and the “OFF” stage indicates the absence of blue light.

FIG. 3 shows graphs illustrating the light-modulated temperature increase and release profile of IgG (Immunoglobulin G) from (A) 0.01% AuNPs-loaded 2% w/w agarose and (B) 20% w/w poloxamer 407 hydrogel composites over period of time.

FIG. 4 is a graph showing a comparison of release rate of different payloads (doxorubicin, lysozyme, bovine serum albumin, Immunoglobulin G, and bevacizumab from AuNPs-loaded agarose hydrogel composite with (r_{ON}) and without (r_{OFF}) blue light exposure (400-500 nm, 508 mW·cm⁻²).

FIG. 5 shows graphs illustrating (A) the effect of gold nanoparticle (AuNPs) concentration on the maximum temperature of the AuNPs/hydrogel composite and the release rate of lysozyme under the exposure of blue light at the intensity of 508 mW·cm⁻² for a polymer composite comprising 2% w/w agarose and 2% w/w/lysozyme and different concentration of gold nanoparticles (AuNPs), and (B) the effect of blue light intensity on the maximum temperature of a AuNPs/hydrogel system (2% w/w agarose, 2% w/w/lysozyme and 1 mg·ml⁻¹ AuNPs) and the release rate of lysozyme.

FIG. 6 shows a graph illustrating the relative bioactivity of released lysozyme from AuNPs/hydrogel composite after blue light exposure using lysis assay of Micrococcus lysodeikticus.

FIG. 7 shows a graph illustrating the relative bioactivity/VEGF binding activity of released Avastin® from an AuNPs/hydrogel composite (with and without 0.1 mg·ml⁻¹ [AuNPs]) after blue light exposure using ELISA of human recombinant VEGF-165.

FIG. 8 shows graphs illustrating the long-term release profile of IgG from hydrogel composite microparticles fabricated using (A) 2% agarose and (B) 4% agarose and different concentrations of gold nanoparticles (AuNPs).

FIG. 9 shows (A) a scanning electron micrograph of the freeze-dried sample of 9% w/w BSA and 0.1% w/w P(OEGMA)-stabilised AuNPs containing PCL (2 KDa) microparticles, and (B) a graph illustrating the release profile of neat \PCL (9% w/w BSA and 0.1% w.w AuNPs) microparticles at different molecular weight (2 KDa, 10 KDa, and 43 KDa) with and without light exposure (10 minutes of 200 mW, 400-500 nm).

FIG. 10 shows graphs illustrating the long-term release study of bevacizumab from 2% agarose hydrogel polymer composite with (A) 0.1 mg·ml⁻¹ stabilised AuNPs and (B) 0.5 mg·ml⁻¹ stabilised AuNPs, in the form of bulk polymer composite, uncoated polymer composite microparticles, and polymer composite microparticles coated with 1% low gelling (LG) agarose, where the concentration of released bevacizumab at each time point was measured before and after the light exposure (ON: 10 minutes of 500 mW, 400-500 nm).

FIG. 11 shows graphs illustrating the bevacizumab real-time release profile under the exposure of blue light (ON) from 2% agarose hydrogel polymer composite with (A) 0.1 mg·ml⁻¹ or (B) 0.5 mg·ml⁻¹ stabilised AuNPs in the form of bulk polymer composite, uncoated polymer composite microparticles, and polymer composite microparticles coated in an injectable 20% w/w poloxamer 407 hydrogel.

FIG. 12 shows a graph comparing samples of 2% agarose, AuNPs (0.05% and 0.01%), AuNPs-loaded 2% agarose hydrogel microparticles and AuNPs-loaded 2% agarose hydrogel microparticles containing 0.25% bevacizumab for in vitro cytotoxicity against ocular cells (HCEC: human corneal epithelial cells, RCE: rabbit corneal endothelial cells, and HRPE: human retinal pigment epithelial cells). The gold nanoparticles (AuNPs) concentration in the 2% agarose-based polymer composite containing 0.25% bevacizumab was prepared at the same concentration as the AuNPs solution only for comparison.

FIG. 13 shows graphs illustrating changes in storage modulus and loss modulus with temperature for four polymer composites that display softening at elevated temperature, whereby the intersection of two tangental lines, represented as dashed lines, reflects the softening point (or Tg) of the thermoplastic polymer matrix in the composite.

FIG. 14 is a graph comparing the glass transition temperature (Tg) as measured by DSC and the softening point (expressed as Tg) as determined by rheometer (DMA) of different polymer matrices in different polymer composites.

FIG. 15 is a graph showing oscillatory shear rheometer measurement of polymer composites containing 18.5% (w/w) poloxamer hydrogel with or without the addition of cross-linking agent (poloxamer 407 diacrylate) and preparing using a photo-curing process.

### DETAILED DESCRIPTION OF THE INVENTION

As used herein, the singular forms “a,” “an,” and “the” designate both the singular and the plural, unless expressly stated to designate the singular only.

The term “about” and the use of ranges in general, whether or not qualified by the term about, means that the number comprehended is not limited to the exact number set forth herein, and is intended to refer to ranges substantially within the quoted range while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

All percentages (%) referred to herein are percentages by weight (w/w or w/v), unless otherwise indicated.

Polymer molecular weights referred to herein are number average molecular weight (Mₙ), unless otherwise indicated.

The present invention relates generally to a polymer composite that is responsive to light and can be triggered to release an agent contained in the composite upon exposure to light.

The fabrication of the polymer composite is simplified by the incorporation of photo-thermal particles and agents dispersed directly in a thermoplastic polymer matrix. This differs from some conventional drug release composites which load the agents subsequent to formation of the composite. The mechanism that modulates the release of an agent to the surrounding environment is triggered by the softening of the thermoplastic polymer matrix, which is caused by the photo-thermal effect of the particles under the exposure of light. The softening results in an increase in the malleability or plasticity of the thermoplastic polymer matrix, which can be detected by changes in storage modulus (which can relate to viscosity changes), such that the diffusion of the agent in the thermoplastic polymer matrix is increased, which then leads to release of the agent from the matrix to the surrounding environment.

In one aspect, the present invention provides a polymer composite for controlled release of an agent comprising:
a thermoplastic polymer matrix;
an agent dispersed in the thermoplastic polymer matrix; and
a plurality of photo-thermal particles dispersed in the thermoplastic polymer matrix, the particles having a non-carboxylic acid stabiliser bound thereto;

wherein the thermoplastic polymer matrix has a softening point when in the composite in the temperature range of from about 30° C. to 70° C. as determined by rheometer, and wherein upon exposure of the composite to photo energy, the particles absorb the photo energy and emit thermal energy to promote the softening of the thermoplastic polymer matrix and modulate the release of the agent from the thermoplastic polymer matrix.

The polymer composite of the present invention comprises a plurality of stabilised photo-thermal particles. In accordance with the invention, the photo-thermal particles are stabilised by having a non-carboxylic acid stabiliser bound to them. The stabilised photo-thermal particles are dispersed in the thermoplastic polymer matrix of the composite and are capable of absorbing photo energy from electromagnetic radiation and converting that photo energy into thermal energy, which is emitted. The thermal energy generated by the photo-thermal particles can be used to heat a thermoplastic polymer matrix and promote softening of the matrix. The softening of the thermoplastic polymer matrix can in turn, trigger or promote the release of an agent that is contained in the matrix by permitting accelerated diffusion of the agent through the softened matrix. The thermoplastic polymer matrix can therefore contain a depot or reservoir of an agent, with release of the agent from that reservoir being modulated by a photo-thermal process that enables the agent to be released on demand at a desired time by exposing the polymer composite to light. The photo-thermal effect for generating heat may be selective, depending on the composition, size and/or shape of the photo-thermal particles and the wavelength of excitation.

Photo-thermal particles suitable for use in the polymer composite of the invention are capable of absorbing electromagnetic radiation at one or more wavelengths. The electromagnetic radiation may be radiation from the ultraviolet, visible and infrared regions of the electromagnetic spectrum. Ultraviolet radiation has a wavelength ranging from about 10 nm to about 380 nm. Visible radiation has a wavelength ranging from about 380 nm to about 700 nm. Infrared radiation has a wavelength ranging from about 700 nm to about 1 mm. It is preferred, however, that the photo-thermal particles absorb radiation having a wavelength in the visible and/or infrared range. In a particular embodiment, the photo-thermal particles may absorb blue and/or green light (approximately 400-600 nm). In another particular embodiment, the photo-thermal particles may absorb near infrared (NIR) light (approximately 700-900 nm).

In one form, the polymer composite of the invention comprises photo-thermal particles that absorb photo-energy having a wavelength in a range selected from the group consisting of from about 10 nm to 1 mm, from about 365 nm to about 1400 nm, and from about 400 nm to about 900 nm.

Photo-thermal particles useful for the polymer composite of the invention may be selected from any one of those that are known to absorb photo energy and be capable of converting the photo energy into heat. In some embodiments, the photo-thermal particles may be metallic particles or carbon particles.

The photo-thermal particles useful for the invention may be in various shapes or forms. In one set of embodiments, the photo-thermal particles may be in the form of rods, spheres, plates, tubes, capsules, hollow shells, dots or colloidal particles.

The photo-thermal particles may further be of any suitable size. In some embodiments, the photo-thermal particles are nanoparticles, which have at least one dimension in the nanometre range. Nanoparticles preferably have a diameter in a range selected from the group consisting of from about 5-400 nm, from about 10-200 nm, from about 20-100 nm, and from about 40-80 nm.

When the polymer composite is used for drug delivery applications, the nanomeric size of the nanoparticles may assist in elimination of the nanoparticles from the body of a subject. The nanomeric size may also help ensure that the optical properties of the polymer composite are not unduly compromised such that the composite remains transparent to light.

In some embodiments, the photo-thermal particles are metallic particles. A variety of metallic particles may be used with the invention. For example, the metallic particles may be selected from the group consisting of gold (Au), silver (Ag), platinum (Pt) and copper (Cu) particles. In some exemplary embodiments, the metallic particles are selected from gold and silver particles. For the purpose of the invention a metallic particle is any particle with a surface that is essentially metallic. A thin oxide or nitride surface layer can exist on the metal surface. As such, a metal inorganic composite, such as a gold coated silica particle, is considered to be a metallic particle for the purpose of the invention.

The photo-thermal particles may be metallic nanoparticles, such as gold nanoparticles or silver nanoparticles. In particular embodiments, the photo-thermal particles are gold nanoparticles. Exemplary gold nanoparticles may be selected from gold nanorods, gold nanospheres and gold nanoshells. Gold nanoparticles may be preferred due to their biocompatibility and their surface plasmon resonance, which exhibits a strong optical extinction in the visible and near-infrared regions (500-900 nm), depending on their size.

Upon light exposure, thermal energy is generated by gold nanoparticles, which can be transferred to the surrounding medium.

In some embodiments, the photo-thermal particles are carbon nanoparticles. The carbon nanoparticles may be selected from the group consisting of carbon nanotubes (single or multiwall), graphene particles, graphene oxide particles and carbon quantum dots.

Photo-thermal nanoparticles, such as metallic nanoparticles or carbon nanoparticles, preferably have a diameter in a range selected from the group consisting of from about 5-400 nm, from about 10-200 nm, from about 20-100 nm, and from about 40-80 nm.

However, the size, shape and/or composition of the photo-thermal particles may be selected based upon its maximum absorption wavelength and the wavelength of the light source that will be absorbed. For example, when using a blue or green light source gold nanoparticles with a diameter of less than 50 nm may be preferred as they have strong absorption in the region 500-550 nm, whereas for an NIR light source, gold nanorods with a diameter of about 100 nm may be selected as they have strong absorption of light around 800 nm.

The polymer composite of the invention may comprise one type of stabilised photo-thermal particle, or it may comprise a mixture of two or more different types of stabilised photo-thermal particles. Mixtures comprising different types of photo-thermal particles may be formed by combining particles of different materials, such as a mixture of metallic particles and carbon particles, or by combining particles of the same material but of different shape and/or size, for example, a mixture of gold nanospheres and gold nanorods.

Gold nanoparticles useful for the present invention may be prepared using a suitable technique. For example, gold nanoparticles may be prepared through the reduction of gold chloride trihydrate (HAuCl₄).

In one set of embodiments, it may be useful for the plurality of photo-thermal particles in the polymer composite to be composed of a mixture of particles of different shape and/or size, as the photo-thermal effect may be modulated by particles of different geometry.

Therefore, the use of photo-thermal particles of different dimensions in the polymer composite may provide a means to control the heating of the thermoplastic polymer matrix after exposure of the composite to light.

The concentration of photo-thermal particles in the polymer composite can vary, and a person skilled in the relevant art may readily determine an appropriate concentration suitable for a particular application. In some embodiments, the polymer composite may comprise from about 0.01 to 10 weight percent of stabilised photo-thermal particles. In some embodiments, the polymer composite comprises stabilised gold nanoparticles in an amount of from about 0.01 to 10.0 mg/ml or 0.001 to 1.0% (w/v).

In accordance with the present invention, the photo-thermal particles have a non-carboxylic acid stabiliser bound to them. The stabiliser assists to inhibit or at least reduce agglomeration of the particles, thereby allowing the particles to be more homogeneously distributed in the thermoplastic polymer matrix. A more homogeneous distribution can help to ensure that localised hot spots do not occur in the thermoplastic polymer matrix, enabling a more even heating of the thermoplastic polymer matrix to be achieved. Without wishing to be limited by theory, it is believed that the stabiliser acts to prevent or reduce agglomeration of the photo-thermal particles. Depending on the composition of the stabiliser and the thermoplastic polymer matrix, the interactions that reduce the agglomeration of the particles may be via steric, electrostatic, intermolecular hydrogen bonding, and/or hydrophobic effects.

The polymer composite of the present invention utilises a non-carboxylic acid stabiliser. By this is meant that the stabiliser does not comprise carboxylic acid groups. It is preferred that non-carboxylic acid stabilisers are used, as stabilisers containing carboxylic acid groups (such as citric acid) may only be capable of forming relatively weak bonds with photo-thermal particles compared to other types of functional groups. Furthermore, carboxylic acid groups can be sensitive to pH, leading to pH dependent colloidal stability of the photo-thermal particles.

Non-carboxylic acid stabilisers used in the polymer composite of the present invention may comprise a functional group selected from the group consisting of a thiol, thiocarbonylthiol, or amino functional group, preferably a thiol functional group. The functional group is preferably a terminal functional group that is capable of interacting with the photo-thermal particle such that the non-carboxylic acid stabiliser can be covalently bound to the photo-thermal particles via that functional group.

In one set of embodiments, the non-carboxylic acid stabiliser may be a non-carboxylic acid polymeric stabiliser or a non-carboxylic acid oligomeric stabiliser.

As used herein, the term “oligomeric stabiliser” denotes a molecule comprising repeat units of relatively low molecular weight. For example PEG is made of repeat units of ethylene oxide, and alkane-thiols are made up of repeat units of [C—C]n. In some cases the oligomeric stabiliser comprises an oligomeric segment, which is formed by polymerising at least one monomer. The monomeric units present in the oligomeric segment may be of one single type, or mixture of two or more different types. An oligomeric stabiliser may comprise an oligomeric segment comprising from 2 to 10 monomeric units.

As used herein, the term “polymeric stabiliser” denotes a macromolecule comprising a polymeric segment formed by polymerising at least one monomer. The polymeric segment comprises more than 10 monomeric units, and is of higher molecular weight than an oligomeric segment. The monomeric units present in the polymeric segment may be of a single type (a homopolymer) or a mixture of two or more different types (a copolymer).

The non-carboxylic acid stabiliser may be bound to the photo-thermal particles by covalent or non-covalent interactions. Non-covalent interactions may be hydrogen bonds or electrostatic interactions. Covalent interactions would generally involve the formation of a covalent bond between the stabiliser and the photo-thermal particle to which it is bound. In one set of embodiments, the non-carboxylic acid stabiliser is covalently bound to the photo-thermal particles.

In one set of embodiments, the non-carboxylic acid stabiliser is a non-carboxylic acid (C₂-C₁₂)-aliphatic molecule comprising a functional group selected from the group consisting of a thiol, thiocarbonylthiol, or amine functional group, preferably a thiol group. In one embodiment, the non-carboxylic acid stabiliser is dodecanethiol.

In one set of embodiments, the non-carboxylic acid stabiliser is a non-carboxylic acid polymeric or oligomeric stabiliser. Non-carboxylic acid polymeric or oligomeric stabilisers useful for the present invention comprise a polymeric or oligomeric segment and are capable of interacting with the thermoplastic polymer matrix to help with the dispersion on the photo-thermal particles in the matrix.

Non-carboxylic acid polymeric or oligomeric stabilisers may also comprise a functional group that is capable of interacting with the photo-thermal particle in order to covalently bond the stabiliser to the photo-thermal particle. In one set of embodiments, the non-carboxylic acid polymeric or oligomeric stabiliser comprises a thiol, thiocarbonylthiol, or amine functional group, preferably a thiol group.

When the photo-thermal particles are gold particles, such as gold nanoparticles, the particles may be stabilised by covalently bonding a thiol terminated polymeric or oligomeric stabiliser to the surface of the particles. Gold nanoparticles have high reactivity with thiol groups, leading to the formation of a covalent sulphur-gold bond. The sulphur-gold bond is a stronger bond that that formed between gold and a carboxylic acid group. Gold nanoparticles may be stabilised via surface functionalisation with a polymeric or oligomeric stabiliser post-synthesis.

The polymeric or oligomeric stabiliser is bound to and may coat at least a portion of the surface of the photo-thermal particles. In some embodiments, the polymeric or oligomeric stabiliser may extend from the surface of the photo-thermal particles.

In one set of embodiments, the non-carboxylic acid polymeric or oligomeric stabiliser may comprise or be a neutral polymer or oligomer. The neutral polymer or oligomer may comprise or be a polyether (high or low molecular weight) or an uncharged polymer or oligomer of an ethylenically unsaturated monomer, such an uncharged polymer or oligomer of an acryloyl or methacryloyl monomer.

Depending on the composition of the polymeric or oligomeric segment, a stabiliser comprising a neutral polymer or oligomer may help to stabilise the photo-thermal particles against agglomeration and aid their retention in the thermoplastic polymer matrix through steric effects, hydrogen bonding interactions and/or hydrophobic effects.

In some embodiments, a stabiliser for the photo-thermal particles is a non-carboxylic acid polymeric stabiliser that comprises or is a neutral polymer.

In some embodiments, the non-carboxylic acid polymeric stabiliser may comprise a neutral polymer selected from the group consisting of poly(ethylene glycol) (PEG), poly(methyl acrylate) (PMA), poly(methyl methacrylate) (PMMA), poly(oligoethylene glycol acrylate) (POEGA) and poly(oligoethylene glycol methacrylate) (POEGMA). Such polymeric stabilisers may also be referred to a neutral polymeric stabiliser.

In one set of embodiments, the non-carboxylic acid stabiliser may comprise or be a charged non-carboxylic acid polymer or oligomer. In such embodiments, the polymer or oligomer may be anionic (i.e. a negatively charged), cationic (i.e. a positively charged) or zwitterionic (i.e. with both negative and positive charge).

The use of a charged molecule for stabilising the photo-thermal particles may be advantageous as the charge may help to inhibit or reduce agglomeration of the particles via electrostatic repulsive effects as well as steric effects. Furthermore, a charged polymeric or oligomeric stabiliser may be capable of interacting with the thermoplastic polymer matrix through electrostatic interactions, which can in turn help with retention of the photo-thermal particles in the thermoplastic polymer matrix.

Reference herein to “positive” or “negative” charge on a polymer or oligomer is intended to mean that the polymer or oligomer contains a moiety or functional group with a positive or negative charge, respectively, with the proviso that negative charges are not provided by a carboxylic acid functional group. The moiety or functional group may of course initially be in a neutral state and subsequently be converted into a charged state. Thus, the functional group or moiety may inherently bear charge, or it may be capable of being converted into a charged state, for example through addition or removal of an electrophile. In the case of a positive charge, the functional group or moiety may have an inherent charge, such as a quaternary ammonium functional group or moiety, or the functional group or moiety per se may be neutral, yet be chargeable to form a cation through, for example, pH dependent formation of a tertiary ammonium cation, or quaternerisation of a tertiary amine group. In the case of negative charge, the functional group or moiety may, for example, comprise an organic acid salt that provides for the negative charge, or the functional group or moiety may comprise an acidic moiety which may be neutral, yet be chargeable to form an anion through, for example, pH dependent removal of an acidic proton.

Where a polymeric or oligomeric stabiliser bears a negative charge, it is a proviso that the negative charge is not provided by a carboxylic acid functional group.

The particular type of polymeric or oligomeric stabiliser used for stabilising the photo-thermal particles may be selected based on the composition of the thermoplastic polymer matrix. For example, a charged polymeric or oligomeric stabiliser may used when the photo-thermal particles are to be dispersed in a polymer matrix with a large number of polar groups, such as hydroxy or amino groups, as electrostatic effects between the polar groups and the charged moiety of the stabiliser can help to reduce agglomeration of the photo-thermal particles in the thermoplastic polymer matrix. In comparison, a neutral polymeric or oligomeric stabiliser may be preferred when the photo-thermal particles are to be dispersed in a thermoplastic polymer matrix, such as polyether and polyester matrix, having fewer (or no) polar groups available for electrostatic interactions.

In one set of embodiments, the non-carboxylic stabiliser is a charged polymeric stabiliser comprising a cationic polymer. Cationic polymers have a net positive charge, which arises from the presence of a positively charged moiety or group in the polymer molecule. Examples of positively charged moieties include phosphonium, sulfonium or quaternary ammonium moieties. Such stabilisers may also be referred to herein as cationic polymeric stabilisers.

In another set of embodiments, the non-carboxylic stabiliser is a charged polymeric stabiliser that comprises an anionic polymer. Anionic polymers have a net negative charge, which arises from the presence of a negatively charged non-carboxylic moiety or group in the polymer molecule. Examples of negatively charged moieties include sulfonic acid moieties. Such stabilisers may also be referred to herein as anionic polymeric stabilisers.

Charged moieties, such as cationic or anionic moieties may be part of a pendant functional group that extends from the main chain of a polymer.

Charged polymeric stabilisers such as anionic and cationic polymeric stabilisers may be formed by polymerising appropriate monomers. In one form, the charged polymer may be prepared using a monomer that contains a functional group or moiety that is in a neutral state and can subsequently converted into a charged state. For example, a cationic polymer may be formed by firstly polymerising a monomer comprising a tertiary amine functional group, which may then be subsequently quaternarised into a positively charged state to form a cationic polymer. In another form, a cationic polymer may be prepared using a monomer that contains a cationic (i.e. positively charged) functional group or moiety. For example, a cationic polymer may be formed from the polymerisation of a monomer comprising a quaternary ammonium functional group. A skilled person would understand that anionic polymers may be prepared in a similar manner with appropriately functionalised monomers.

A cationic polymeric stabiliser will present a net positive charge. Generally at least about 10%, or at least 30%, or at least 40%, or at least 50%, or at least 70%, or at least 90%, or all of the polymerised monomer residue units that make up the cationic polymeric moiety of the stabiliser comprise a positive charge.

Non-carboxylic acid polymeric stabilisers as described herein (such as cationic, anionic or neutral polymeric stabilisers) may, and preferably will, also comprise a non-carboxylic acid terminal functional group, which is capable of reacting with a photo-thermal particle (such as a gold nanoparticle) to aid in the covalent attachment of the stabiliser to a photo-thermal particle. Preferably, the terminal functional group is a thiol functional group.

A polymeric stabiliser useful for stabilising the photo-thermal particles may be prepared by any suitable means. The polymeric stabiliser may be an anionic, cationic or neutral non-carboxylic acid polymeric stabiliser, as described herein.

Polymeric stabilisers (such as cationic, anionic and neutral polymeric stabilisers) described herein may comprise any suitable number of polymerised monomer units in the polymeric segment of the stabiliser. In some embodiments, the polymeric stabiliser comprises from about 5 to about 200, or about 40 to about 200, or about 80 to about 200 polymerised monomer units.

In one embodiment, polymeric stabilisers as described herein are prepared by polymerisation of ethylenically unsaturated monomers. Polymerisation of the ethylenically unsaturated monomers is preferably conducted using a living polymerisation technique.

Living polymerisation is generally considered in the art to be a form of chain polymerisation in which irreversible chain termination is substantially absent. An important feature of living polymerisation is that polymer chains will continue to grow while monomer and reaction conditions to support polymerisation are provided. Polymer chains prepared by living polymerisation can advantageously exhibit a well defined molecular architecture, a predetermined molecular weight and narrow molecular weight distribution or low polydispersity. Another advantage of living polymerisation is the end group of the resultant polymer chain can be retained, spatially controlled, and modified to provide an anchoring functional group that is capable of anchoring the polymeric stabiliser to the surface of photo-thermal particles. For instance, thiol terminal groups can be utilised to attach to gold nanoparticles or for attachment of other agents.

Examples of living polymerisation include ionic polymerisation and controlled radical polymerisation (CRP). Examples of CRP include, but are not limited to, iniferter polymerisation, stable free radical mediated polymerisation (SFRP), atom transfer radical polymerisation (ATRP), and reversible addition fragmentation chain transfer (RAFT) polymerisation.

Equipment, conditions, and reagents for performing living polymerisation are well known to those skilled in the art.

Where ethylenically unsaturated monomers are to be polymerised by a living polymerisation technique, it will generally be necessary to make use of a so-called living polymerisation agent. By “living polymerisation agent” is meant a compound that can participate in and control or mediate the living polymerisation of one or more ethylenically unsaturated monomers so as to form a living polymer chain (i.e. a polymer chain that has been formed according to a living polymerisation technique).

Living polymerisation agents include, but are not limited to, those which promote a living polymerisation technique selected from ionic polymerisation and CRP.

In one embodiment, a polymeric stabiliser useful for the present invention is prepared by CRP.

In one embodiment, the polymeric stabiliser is prepared by RAFT polymerisation. A polymer formed by RAFT polymerisation may conveniently be referred to as a RAFT polymer. By virtue of the mechanism of polymerisation, such polymers will comprise a residue of the RAFT agent that facilitated polymerisation of the monomer.

Polymers prepared by RAFT polymerisation may be desirable for use a polymeric stabiliser in the invention as the resulting polymer can bear a dithioester end group, which is a residue from the RAFT agent used to form the polymer. The dithioester end group can be reduced to a thiol end group under appropriate conditions. This provides a convenient route for producing a terminal thiol functional group.

However, a skilled person would appreciate that polymers prepared using other living polymerisation techniques can have an end group that can be modified to a terminal thiol group using conventional chemical techniques.

The thiol terminal group of the polymer can covalently react with photo-thermal particles such as gold nanoparticles. This results in covalent bonding of the polymer to the photo-thermal particles, thus allowing the polymer to act as a stabiliser to reduce or inhibit agglomeration of the photo-thermal particles when the particles are dispersed in the thermoplastic polymer matrix of the polymer composite.

A range of RAFT agents may be used to prepare a RAFT polymer. Preferably, the RAFT agent comprises a thiocarbonylthio group (which is a divalent moiety represented by the group: —C(S)S—). Examples of RAFT agents are described in Moad G.; Rizzardo, E; Thang S, H. Polymer 2008, 49, 1079-1131 and Aust. J. Chem., 2005, 58, 379-410; Aust. J. Chem., 2006, 59, 669-692; Aust. J. Chem., 2009, 62, 1402-1472 (the entire contents of which are incorporated herein by reference). A non-limiting example of a RAFT agent that can be used is 2-cyano-2-propyl benzodithioate. A person skilled in the relevant art would be able to select other RAFT agents that could be used to prepare suitable polymers.

Some RAFT polymers can interact with gold nanoparticles without cleavage of the RAFT end group and thus the RAFT polymer could be used to stabilise gold nanoparticles that are used as photo-thermal particles (see for example: A.-S. Duwez, P. Guillet, C. Colard, J.-F. Gohy and C.-A. Fustin, Macromolecules, 2006, 39, 2729-2731; C.-A. Fustin and A.-S. Duwez, J. Electron. Spectrosc. Relat. Phenom., 2009, 172, 104-106.).

Ethylenically unsaturated monomers that can be polymerised to form a non-carboxylic acid polymeric stabiliser may be hydrophilic, such that the resulting polymer may suitably be a hydrophilic polymer. Polymeric stabilisers comprising a hydrophilic polymeric segment may be desirable when the photo-thermal particles are to be dispersed in a hydrophilic thermoplastic polymer matrix, such as a thermoplastic hydrogel.

In other embodiments, ethylenically unsaturated monomers that may be polymerised to form a non-carboxylic acid polymeric stabiliser may be amphiphilic or hydrophobic, such that the resulting polymer may be an amphiphilic or hydrophobic polymer. Polymeric stabilisers comprising an amphiphilic or hydrophobic polymeric segment may be desirable when the photo-thermal particles are to be dispersed in a hydrophobic thermoplastic polymer matrix.

Examples of ethylenically unsaturated monomers include allyl monomers, vinyl monomers, styrenyl monomers, acryloyl and methacryoyl monomers such as acrylate and methacrylate ester monomers, acrylamido, and methacrylamido monomers, mixtures of these monomers, and mixtures of these monomers with other monomers.

Ethylenically unsaturated monomers employed to form the non-carboxylic acid polymeric stabiliser may comprise a pendant group that is charged or uncharged. A proviso that a charged group is not a carboxylic acid group Depending on the nature of the pendant group, the polymer resulting from the polymerisation of the ethylenically unsaturated monomer may be charged (i.e. cationic, anionic or zwitterionic) or uncharged (i.e. neutral).

In one set of embodiments, the polymeric stabiliser comprises a cationic polymer comprising a pendant quaternary ammonium group. The cationic polymer may be a polymer of a cationic monomer, such as an ethylenically unsaturated monomer bearing a quaternary ammonium group.

Some examples of ethylenically unsaturated monomers that may be used in preparing a cationic polymer include, but are not limited to, 2-aminoethyl methacrylate hydrochloride, N-[3-(N,N-dimethylamino)propyl] methacrylamide, N-(3-aminopropyl)methacrylamide hydrochloride, N-[3-(N,N-dimethylamino)propyl]acrylamide, N-[2-(N,N-dimethylamino)ethyl]methacrylamide, 2-N-morpholinoethyl acrylate, 2-N-morpholinoethyl methacrylate, 2-(N,N-dimethylamino)ethyl acrylate, 2-(N,N-dimethylamino)ethyl methacrylate, 2-(N,N-diethylamino)ethyl methacrylate, 2-acryloxyyethyltrimethylammonium chloride, 2-methacryloxyyethyltrimethylammonium chloride, methacrylamidopropyltrimethylammonium chloride, 2-(tert-butylamino)ethyl methacrylate, diallyldimethylammonium chloride, 2-(diethylamino)ethylstyrene, 2-vinylpyridine, and 4-vinylpyridine, and combinations thereof.

In one form, the cationic polymer is a polymer of the cationic monomer, methacryloyloxyethyl trimethyl ammonium chloride. In such embodiments, the stabiliser may be a polymeric stabiliser comprising poly(methacryloyloxyethyl trimethyl ammonium chloride).

In one set of embodiments, the polymeric stabiliser comprises an anionic polymer comprising a pendant organic acid group. The anionic polymer may be a polymer of an anionic monomer, such as an ethylenically unsaturated monomer bearing an organic acid group.

Some examples of ethylenically unsaturated monomers that may be used in preparing an anionic polymer include, but are not limited to, sulfonic acids such as methacryloyloxypropylsulfonic acid, vinylsulfonic acid and p-styrenesulfonic acid, and their salts and combinations thereof.

In one set of embodiments, the polymeric stabiliser comprises a zwitterionic polymer, which comprises positive and negative charges substantially in balance with other. A zwitterionic polymer may be a polymer of an ethylenically unsaturated monomer having a pendant group bearing both negative and positive moieties.

Some examples of ethylenically unsaturated monomers that may be used in preparing a zwitterionic polymer include, but are not limited to, N-(3-sulfopropyl)-methacroyloxyethyl-N,N-dimethylammonium-betaine, N-(3-sulfopropyl)-N-methacrylamidopropyl-N,N-dimethylammonium-betaine, 1-(3-sulfopropyl)-2-vinyl-pyridinium-betaine and 2-(methacryloyloxy)ethyl-2-(trimethylammonium) ethylphosphate.

In one set of embodiments, the polymeric stabiliser comprises a neutral polymer. The neutral polymer may be a polymer of an ethylenically unsaturated monomer having a pendant group bearing no charged moieties. Some examples of ethylenically unsaturated monomers that may be used in preparing a neutral polymer include, but are not limited to esters of acryloyl and methacryloyl monomers, such as (C₁-C₄) esters of acryloyl or methacryloyl monomers (for example, methyl acrylate, methyl methacrylate, ethyl acrylate and ethyl methacrylate) and (oligoethylene glycol) esters of acryloyl and methacryloyl monomers. In some embodiments, the (oligoethylene glycol) esters of acryloyl and methacryloyl monomers may comprise from between 5 to 87 ethylene glycol units.

In one form, the neutral polymer is a polymer of the monomer (oligoethylene glycol methyl ether) methacrylate. In such embodiments, the stabiliser may be a polymeric stabiliser comprising poly(oligoethylene glycol methyl ether methacrylate) (P(OEGMA)). A skilled person would recognize that the oligo(ethylene glycol) pendant group of the monomer and the resulting polymer would be a neutral group.

As described herein, the polymer composite of the invention comprises a thermoplastic polymer matrix.

The thermoplastic polymer matrix is generally a two-dimensional or three-dimensional matrix comprising at least one thermoplastic polymer.

The term “thermoplastic” as used with reference to a polymer matrix or material described herein refers to a thermomechanical property of the thermoplastic polymer matrix or material, which means that the thermoplastic polymer matrix or material is responsive to heat. In the present invention, the heat is generated upon photo-thermal particles present in the polymer composite being exposed to light. The thermoplastic polymer matrix is capable of becoming more plastic (i.e. softer, less viscous, more pliable or more malleable) when subjected to an increase in temperature. Upon removal of the heat (i.e. by ceasing exposure of the composite to light) the thermomechanical properties allow the thermoplastic polymer matrix or material to revert to a more viscous or solidified or hardened state as the thermoplastic polymer matrix or material is cooled. Thermoplastic behaviour characterised by softening with an increase in temperature can be ascertained by determining changes in the storage modulus of the thermoplastic polymer matrix as heat is applied.

The softening point of the thermoplastic polymer matrix in the composite can be determined by dynamic mechanical analysis (DMA) using a rheometer, for example, an oscillatory shear rheometer. Using such techniques, softening can be indicated by a decrease in the storage modulus (E′ or G′) and loss modulus (E″ and G″) of the polymer matrix with an increase in temperature.

The softening point of a polymer is often referred to as the glass transition temperature (Tg). Thermoplastic polymer matrices useful for the polymer composite of the invention may have a Tg within the range of from about 30° C. to 70° C.

The measurement of storage and loss modulus is described in various ASTM standards, such as D7028 (for measuring Tg), E1640 (Tg of amorphous and semi-crystalline materials), D4065 (loss moduli) and D4440 (rheology for small samples). Depending on the nature of the test sample, the softening point of the thermoplastic polymer matrix can be determined in accordance with procedures described in any one of these standards.

Preferred rheological techniques for measuring the softening point of the thermoplastic polymer matrix in the polymer composite in the desired temperature range are described in standards such as ASTM D4065, D5279, D7028, E1640 and D4440.

In one embodiment, the softening temperature is preferably determined in accordance with ASTM E1640. This standard describes a procedure for determining glass transition temperature (Tg) by dynamic mechanical analysis (DMA). In accordance with this procedure, Tg is taken as the extrapolated onset to the sigmoidal change in the storage modulus observed when a material changes from hard or brittle to soft and rubbery. The construction of two tangential lines to the storage modulus curve and determination of the temperature at which these tangential lines intersect provides the Tg.

The softening point of the thermoplastic polymer matrix reflects a change in the matrix to a softer, more rubbery, or more molten state as characterised by a decrease in storage modulus. Thus procedures for measuring Tg can also be used to determine the softening point of the polymer matrix.

In some embodiments of the polymer composite of the invention, softening of the thermoplastic polymer matrix in the composite commences when the temperature of the composite is within the range of from about 30° C. to 70° C. The softening can start to occur when thermal energy emitted by photo-thermal particles within the composite raises the temperature to within the desired temperature range.

The thermoplastic polymer matrix is responsive to the heat or thermal energy that is emitted by the photo-thermal particles when the polymer composite is exposed to light. The heat softens the thermoplastic polymer matrix in the composite and typically this may be detected as a decrease in the storage modulus of the matrix.

In accordance with the requirements of the invention, the thermoplastic polymer matrix softens in the polymer composite at the onset of the softening point, being in the temperature range of from about 30° C. to 70° C. This means that when the polymer composite per se is heated at a temperature in the range of from 30° C. to 70° C., then the thermoplastic polymer matrix forming a part of the polymer composite is capable of softening and transforming into more malleable or molten (i.e. less viscous) state within the composite at that temperature. The softening point is determined by dynamic mechanical analysis, which involves measuring the storage modulus by rheometer and determining the transition point at which the storage modulus decreases.

Other components of the polymer composite, such as the stabilised photo-thermal particles, the agent to be released and any solvent, are also present in the composite when tests are performed to determine the softening point and thus the temperature at which softening of the thermoplastic polymer matrix occurs. A skilled person would appreciate that other components present in the polymer composite might influence the thermomechanical behaviour of the thermoplastic polymer matrix. Neat polymer matrices (not in the composite) that would usually soften at a temperature outside the range of from 30° C. to 70° C. have been found to soften at a temperature that falls within the specified temperature range when incorporated as part of the composite due to the effect of the other components in the composite. Thus it is important that the test carried out to determine the softening point of the thermoplastic polymer matrix and the temperature at which this occurs is performed when the thermoplastic polymer matrix is a part of the polymer composite.

When the polymer composite is used for biomedical applications such as drug delivery, it can be desirable for the thermoplastic polymer matrix to soften in the composite at a temperature that is compatible with biological tissue. This might be particularly advantageous when the polymer composite forms part of a device designed to be in contact with or implanted in the body of a subject, such as a human or animal subject. A temperature that is compatible with biological tissue may have minor or minimal adverse effects on biological tissue, such as cellular tissue. For example, the temperature may cause minor or minimal damage to biological tissue that contacts, surrounds or is located nearby the polymer composite.

For instance, for biomedical applications it is important that the thermoplastic polymer matrix have a softening point when in the polymer composite in a temperature range of from about 30° C. to 70° C. as this is a clinically acceptable temperature range. It is important that the softening point is not too high (i.e. above 70° C.) otherwise cell death may occur. Examples of thermoplastic polymers that are not suitable for the polymer composite of the invention include polystyrene (with a Tg of 100° C.), polymethyl methacrylate (with a Tg of 115° C.), polypyrrole (with a Tg of 97° C.) and polydimethyl acrylamide (with a Tg of 89° C.). Incorporation of such polymers with high Tg values into the thermoplastic polymer matrix of the composite would not result in a polymer matrix having a softening point in the temperature range of 30° C. to 70° C.

It is also important that the softening point is not too low (i.e. below 30° C.) or else the agent contained in the thermoplastic polymer matrix may be released too quickly. Furthermore, it can be important for the polymer composite of the invention that the softening point of the polymer matrix is not below 30° C. to help ensure that the polymer composite remains solid enough or stiff enough to have appropriate physical characteristics to allow it be handled for implantation or for injection when at ambient room temperature. It would be appreciated that some thermoplastic polymer matrices can start to soften at low temperature (less than 30° C.) and continue to soften as temperature is increased above 30° C. However, these polymer matrices may impart detrimental effects on the handling properties of polymer composites containing them. As a result, in one embodiment it can be preferred that such polymer matrices are not utilised in the composites of the invention.

In some embodiments, the thermoplastic polymer matrix may have a softening point when in the composite at a temperature that is approximately at or above physiological temperature, while also being lower that which will adversely affect biological tissue. In particular embodiments, the thermoplastic polymer matrix may have a softening point when in the composite within a temperature range of from 35° C. to 65° C., or in a range of from 37° C. to 60° C., or in a range of from 40° C. to 55° C.

In some embodiments, the thermoplastic polymer matrix may begin to soften in the composite at a temperature in a range of from the group consisting of from 30° C. to 70° C., from 35° C. to 65° C., from 37° C. to 60° C., or from 40° C. to 55° C.

A thermoplastic polymer matrix that has a softening point when in the composite at a temperature in a range of from 30° C. to 70° C., from 35° C. to 65° C., from 37° C. to 60° C., or from 40° C. to 55° C., may also be advantageous where the agent to be released from the composite is a drug. A number of drugs may be sensitive to temperature and in the case of biopharmaceuticals, might be at risk of deactivation or degradation if exposed to high temperature. The present invention may therefore allow the pharmacological efficacy of an agent such as a drug to be substantially preserved.

The photo-thermal particles present in the polymer composite convert photo-energy into thermal energy. The thermal energy heats the thermoplastic polymer matrix, leading to softening of the thermoplastic polymer matrix as a result of having reached the softening point in the desired temperature range, thereby modulating the release of the agent contained in the matrix to the surrounding environment. By “softening” of the thermoplastic polymer matrix is meant that the thermoplastic polymer matrix in the composite becomes less viscous and thus more pliable or malleable when the composite is heated in the desired temperature range.

The thermoplastic (softening) property of thermoplastic polymer matrix is determined by storage modulus changes and the resulting onset of softening at the softening point, which is observed in the temperature range of from about 30° C. to 70° C. The reduction in storage modulus is detected using a rheometer, as this technique can measure the change in storage modulus of the polymer matrix directly as temperature is increased. The rheometer can also measure changes in the viscosity of the polymer matrix as temperature is increased, which can be correlated with changes in storage modulus if desired.

Accordingly the thermoplastic polymer matrix exhibits a softening point when in the range of from about 30° C. to 70° C. As used herein the term “softening point” refers to the temperature at which the storage modulus of the thermoplastic polymer matrix starts to change (i.e. decrease), as determined by rheometer. The decrease in storage modulus characterising the softening point of the polymer matrix can be observed as an abrupt change or as a more gradual change in modulus. The softening point can be measured in accordance with internationally accepted standards as described herein, such as ASTM E1640.

Polymers that are capable of softening in the composite and exhibit the desired softening point may also possess a thermal property, such as a glass transition temperature (Tg) or melting temperature (Tm), within the desired temperature range. The glass transition temperature (Tg) or melting temperature (Tm) is indicative of a change in the physical state of the polymer in response to temperature, and can give an indication as to whether a given thermoplastic polymer matrix is likely to have a softening point and thus commence softening in the polymer composite in the desired temperature range. Thermal phase transitions such as Tg and Tm may be determined by techniques known in the art. For example, differential scanning calorimetry (DSC) and dynamic mechanical thermal analysis (DMTA) may used.

A thermoplastic polymer matrix may also be modified to enable it to have a softening point in the desired temperature range. Modifications may be via the incorporation of additives (e.g. plasticisers, salts, cross-linking agent etc) in the thermoplastic polymer matrix or by fabricating the thermoplastic polymer matrix (and hence polymer composite) into different shapes or forms such as microspheres and the like.

The thermoplastic polymer matrix comprises at least one polymer. The polymer is generally a thermoplastic polymer. The polymer present in the thermoplastic polymer matrix may be of a suitable architecture, such as a linear, branched, interpenetrating network or crosslinked architecture (both covalent and non-covalent cross-linking).

In some embodiments, the thermoplastic polymer matrix comprises a linear polymer. The linear polymer may be a natural polymer or a synthetic polymer. Natural polymers are obtained or derived from sources found in nature. Synthetic polymers may be homopolymers or copolymers formed through the polymerisation of suitable monomers. Copolymers may be random, alternating, block or graft copolymers.

In some embodiments, the thermoplastic polymer matrix comprises a crosslinked polymer. A crosslinked polymer has a three-dimensional network structure and may comprise chains of a natural and/or synthetic polymer crosslinked via covalent or non-covalent bonds. In some instances, cross-linking may be mediated by cross-linking agents that link together different polymer chains via covalent bonds.

The thermoplastic polymer matrix may suitably comprise at least one linear or crosslinked thermoplastic polymer that softens at a temperature range of from about 30° C. to 70° C. as determined by rheometer.

The softening of the thermoplastic polymer matrix modulates the release of an agent from the polymer matrix. Modulation of release involves a change in the rate of release of the agent. In some embodiments, modulation of release can involve an acceleration of the diffusion and release of agents such as drugs from the thermoplastic polymer matrix. This accelerated release generally occurs when the polymer composite is exposed to light. Release of the agent ceases or slows when the light source is removed due to solidification of the thermoplastic polymer matrix as the matrix cools. In some embodiments, modulation of release can involve triggering the diffusion and release of agents such as drugs from the thermoplastic polymer matrix.

In addition to possessing the requisite thermoplastic behaviour, it can be desirable for the thermoplastic polymer matrix to also be biocompatible and/or biodegradable. This may be advantageous when the polymer composite of the invention is used for drug delivery applications where it is important for the composite to be compatible with a biological environment. For drug delivery applications, biocompatible and/or biodegradable thermoplastic polymer matrices may be selected from those that are generally recognised as safe (GRAS) and are suitable for biomedical applications. Suitable thermoplastic polymer matrices may be obtained from commercial sources.

The term “biocompatible” as used herein with reference to a compound or material means that the compound or material is minimally toxic or non-toxic to a biological environment, such as living tissue or a living organism.

The term “biodegradable” as used herein with reference to a compound or material means that the compound or material is capable of being broken down or decomposed in a biological environment.

In order to ensure that the photo-thermal particles in the polymer composite can absorb photo energy to facilitate the photo-thermal effect, it may further be desirable for the thermoplastic polymer matrix to be substantially transparent for a given wavelength of electromagnetic radiation. In one set of embodiments, the thermoplastic polymer matrix is substantially transparent to infra-red radiation and/or visible light.

In one set of embodiments, the thermoplastic polymer matrix is in the form of a hydrogel comprising a polymer phase and an aqueous liquid phase. Thus the thermoplastic polymer matrix may comprise a hydrogel. Hydrogels are generally a class of polymers composed of a three-dimensional polymer network solvated by an aqueous liquid phase. The polymer network may be held together via crosslinks formed with covalent bonds or non-covalent bonds. A thermoplastic polymer matrix in the form of a hydrogel may be beneficial as it can contain a high aqueous liquid content, making it compatible with biological systems. The solids (polymer) content of the hydrogel may be adjusted to suit a selected polymer and desired application.

The hydrogel may also be porous, which allows compounds and other materials to flow through the hydrogel. The porosity may be provided by interconnected channels or pores within the hydrogel.

In accordance with the requirements of the invention, a thermoplastic polymer matrix comprising a hydrogel has a softening point in the polymer composite at a temperature in a range selected from the group consisting of from 30° C. to 70° C., from 35° C. to 65° C., from 37° C. to 60° C., and from 40° C. to 55° C., as determined by rheometer.

In one set of embodiments, the polymer phase of the hydrogel comprises a hydrophilic polymer. The hydrophilic polymer may be selected from the group consisting of a polysaccharide, a polypeptide, polyether, poloxamer (Pluronic®), polyester, poly(vinyl pyrrolidone), poly(ethylene-vinyl acetate) and poly(vinyl alcohol). The hydrophilic polymer will generally form the polymer phase of the hydrogel that has thermoplastic properties.

The hydrophilic polymer used in the hydrogel may exhibit a phase transition, such as a glass transition (Tg) or melting temperature (Tm) at a temperature in the range of from about 30° C. to 70° C. For example, polyethers such as poly(ethylene glycol), poloxamers, and polyvinylpyrrolidone exhibit glass transition or melting temperatures between 30-70° C. As explained above, this could help indicate whether a hydrogel containing the hydrophilic polymer would have a softening point and thus be capable of softening in the polymer composite in the desired temperature range. In some embodiments, the hydrophilic polymer exhibits a phase transition temperature in a range of from 35° C. to 65° C., from 37° C. to 60° C., or from 40° C. to 55° C.

In some embodiments, once the softening point of the thermoplastic polymer matrix has been reached, the polymer matrix continues to soften with increasing temperature. With further increases in temperature, a melting point (Tm) for the polymer matrix can be reached. A skilled person would appreciate that the polymer matrix transitions from solid to liquid form at the melting point. In some embodiments it can be desirable to avoid thermoplastic polymer matrices that undergo significant melting in the temperature range of from 30° C. to 70° C. as this may lead to disintegration of the polymer composite and thus render the polymer composite incapable of providing for modulated release of the agent contained therein. In some embodiments, this may be achieved by utilising thermoplastic polymer matrices having a melting point (Tm) that is higher than the temperature produced due to the thermal energy emitted by the photo-thermal particles.

A skilled person would understand that hydrogels are generally formed by dissolving a desired quantity of one or more hydrogel-forming polymers into an aqueous solvent to form a solution. Generally, the aqueous solvent is water. The aqueous solution used to form the hydrogel may comprise other optional additives, such as salts or cross-linking agents, if desired, to facilitate hydrogel formation.

The aqueous solution used to form the hydrogel will also generally comprise the polymeric or oligomeric stabilised photo-thermal particles and the agent that is to be released from the polymer composite. In this manner, the polymer composite of the invention may be readily prepared by subjecting the aqueous solution to conditions allowing the hydrogel to form.

The desired quantity of hydrogel-forming polymer will depend on the type of polymer that is used, the desired solids content and the properties required of the resultant hydrogel. For instance, hydrogels with higher solids content (i.e. more polymer) may be stiffer and less elastic than hydrogels formed from solutions containing lower quantities of polymer.

Hydrogels used as thermoplastic polymer matrices for the polymer composite of the invention may exhibit reversible sol-gel behaviour. Such hydrogels can be in the form of a solid or semi-solid gel state at room temperature (approximately 20° C.), then soften or melt to a liquid or sol state as temperature increases and revert to the solid state upon cooling. In one form, hydrogels with reversible sol-gel behaviour may begin to soften or melt at a temperature in a range of from about 30° C. to 70° C., from about 35° C. to 65° C., from about 37° C. to 60° C., or from about 40° C. to 55° C.

Hydrophilic polymers that can be employed in forming hydrogels useful in this invention include polysaccharides, polypeptides polyethers, poloxamers (Pluronic®), polyesters, poly(vinyl pyrrolidone), poly(ethylene-vinyl acetate) and poly(vinyl alcohol), which may form aggregates in solution, and which are bonded through non-covalent interactions such as hydrogen bonds, electrostatic interactions or physical entanglements. The aggregates form the structure of the three-dimensional network of the hydrogel polymer phase.

In one set of embodiments, the hydrogel comprises a polysaccharide. Polysaccharides are polymeric carbohydrate molecules composed of saccharide units linked together by glycosidic linkages. In one set of embodiments, the polymer phase of the hydrogel may comprise a polysaccharide selected from the group consisting of agarose, carrageenan, chitosan, gellan gum, starch, alginate, hyaluronic acid, dextran, cellulose, and mixtures thereof.

In a particular embodiment, the hydrogel comprises agarose. Accordingly, in such embodiments, the thermoplastic polymer matrix is an agarose hydrogel. Agarose may advantageously mimic the mechanical properties of biological tissue.

Agarose is a non-toxic and biocompatible hydrophilic polymer that forms a non-covalently bonded gel at temperatures of approximately 30 to 42° C. depending on the type and concentration of the agarose. The agarose gel can soften above 40° C. and melt at temperatures above 65° C.

For example, agarose hydrogels comprising 2% and 4% w/w agarose exhibit softening temperatures of 45° C. and 50° C. respectively, when measured by rheometry. They also have glass transition temperatures (Tg) of approximately 54° C. and 58° C. respectively as determined by differential scanning calorimetry (DSC).

In some embodiments, when the thermoplastic polymer matrix is an agarose hydrogel, the agarose hydrogel may comprise from between 0.1 to 20% (w/w) agarose as the polymer phase, with the remaining 80% to 99.9% (w/w) being the aqueous liquid phase. In one set of embodiments, an agarose hydrogel may comprise from between 0.5 to 10% (w/w) or 1 to 5% (w/w) agarose as the polymer phase.

In one set of embodiments, the hydrogel comprises a polypeptide. An exemplary polypeptide is gelatin. Gelatin is soluble in aqueous solvents at elevated temperature. The aqueous solution can set to a gel upon cooling to room temperature. In some embodiments, when the thermoplastic polymer matrix is a gelatin hydrogel, the gelatin hydrogel may comprise from between 0.1 to 10% (w/w) gelatin as the polymer phase. The gelatin may be optionally crosslinked with cross-linking agents such as glutaraldehyde or succinic acid to increase the modulus or stiffness of the hydrogel.

In one set of embodiments, the hydrogel comprises a poloxamer or Pluronic®. Poloxamers are a family of biocompatible ABA block copolymers that are composed of two hydrophilic poly(ethylene oxide) blocks (A) and a hydrophobic poly(propylene oxide) block (B). When poloxamers are heated above a critical temperature (the critical gelation temperature), the polymer is able to form a gel through micellization of the polymer and physical entanglement and packing of the micelle structures. Gelation of the poloxamer is characterised by an increase in storage modulus for a solution containing the polymer. Upon further heating to temperatures above the critical gelation temperature, the poloxamer hydrogel then softens, which can be detected by observing a decrease in storage modulus for the gel.

The unique thermomechanical properties of poloxamers, whereby they gel upon being heated and then soften when further heat is applied, can be utilized to introduce an aqueous solution containing the poloxamer, the stabilised photo-thermal particles, the agent to be released, and any other optional components, to a body site of a patient by injection. The injected solution would then solidify to form a hydrogel containing the photo-thermal particles and agent at physiological temperature (e.g. 37° C.). When the temperature of the hydrogels is further increased through heating via the photo-thermal effect, the hydrogel then softens to provide modulated release the agent from the polymer composite. As explained herein, the softening of the hydrogel in the polymer composite may be observed as a decrease in the storage modulus of the hydrogel, which can be characterised by rheometer as the thermal phase transition. The storage modulus of the poloxamer-based hydrogels can be increased modifying the poloxamer to contain crosslinkable groups that may be crosslinked by a photo-curing process at 37° C., while still maintaining the softening behaviour at elevated temperature.

In one set of embodiments, the thermoplastic polymer matrix comprises a hydrogel, where said hydrogel comprises a poloxamer selected from poloxamer 407 (also known as Pluronic® F127), poloxamer 338 (also known as Pluronic® F108), and poloxamer 237 (also known as Pluronic® F87).

When the thermoplastic polymer matrix is a poloxamer hydrogel, the hydrogel may comprise from between 1 to 50% (w/w) poloxamer as the polymer phase, with the remaining 50% to 99% (w/w) being the aqueous liquid phase. A person skilled in the relevant art would appreciate the concentration of poloxamer in the hydrogel might depend on the type of poloxamer used. In some instances, the hydrogel may comprise more than 10% (w/w) or more than 20% (w/w) poloxamer, to enable the polymer to form a gel at physiological temperature.

In one set of embodiments, the hydrogel may comprise a poloxamer comprising at least one crosslinkable group. The crosslinkable group can undergo crosslinking under suitable conditions to facilitate formation of a crosslinked polymer. Crosslinking may be via covalent or non-covalent crosslinking. In one embodiment, crosslinkable groups may be ethylenically unsaturated groups, which are capable of undergoing covalent crosslinking. Examples of ethylenically unsaturated groups include but are not limited to vinyl, acrylate and methacrylate groups.

A poloxamer comprising at least one crosslinkable group may be present in the hydrogel in addition to, or in place of, a conventional poloxamer.

In one set of embodiments, the thermoplastic polymer matrix comprises a crosslinked hydrogel. The crosslinked hydrogel may comprise a crosslinked poloxamer. The crosslinked poloxamer may be formed by covalently crosslinking a convention poloxamer with a poloxamer comprising at least one crosslinkable group, such as an ethylenically unsaturated group.

In one set of embodiments the thermoplastic polymer matrix comprises a thermoplastic polymer in neat form. The term “neat” as used herein with reference to a polymer indicates that the polymer is not solvated or hydrated.

A thermoplastic polymer matrix will comprise at least one neat thermoplastic polymer having the requisite thermomechanical properties described herein, which allows the thermoplastic polymer matrix to soften in the polymer composite at a temperature in the range of from 30° C. to 70° C.

The use of a neat thermoplastic polymer in the thermoplastic polymer matrix may be preferred for some applications as it may allow the polymer composite to be fabricated into a wider variety of shapes compared to hydrogel polymers. Furthermore, a polymer composite having a thermoplastic polymer matrix comprising a neat polymer may also have greater storage stability.

The neat thermoplastic polymer may be porous or non-porous.

In some embodiments, the neat thermoplastic polymer is porous. In such embodiments, the neat polymer may comprise one or more pores or openings. The average size of the pores or openings may be in the range of from about 100 nm to 5000 nm.

A porous neat thermoplastic polymer may be prepared through the evaporation of solvent after fabrication of a polymer composite of the invention using suitable emulsion techniques.

The thermoplastic polymer matrix may comprise at least one neat thermoplastic polymer selected from the group consisting of polyesters, polyamides, polyethers, poly(vinyl pyrrolidone), poly(ethylene-vinyl acetate), polyoxazoline and mixtures thereof.

In one set of embodiments, the thermoplastic polymer matrix comprises a polyester in neat form. The neat polyester may be a homopolymer or copolymer of at least one monomer selected from the group consisting of ε-caprolactone, lactic acid, glycolic acid, lactide and glycolide. A benefit associated with a thermoplastic polymer matrix comprising a polyester formed with these monomers is that the resulting polyester is biocompatible and biodegradable and would be capable of degrading in vivo to non-toxic degradation products.

Polyesters that are neat polymers of at least one monomer selected from ε-caprolactone, lactic acid, glycolic acid, lactide, glycolide and combinations thereof, can melt at a relatively low temperature, leading to softening or melting of the polymer at a temperature in a range of from about 30° C. to 70° C. In some embodiments, the neat polyester has a softening point in the composite in a temperature range of from 35° C. to 65° C., from 37° C. to 60° C., or from 40° C. to 55° C. The softening point of the neat polymer is determined by rheometer, as described herein.

Polyesters that are neat polymers of at least monomer selected from ε-caprolactone, lactic acid, glycolic acid, lactide, glycolide and combinations thereof can further exhibit reversible sol-gel behaviour, where the polymer transforms from a solid at room temperature (approximately 20° C.) to a molten or liquid state at elevated temperature (e.g. at a temperature between 30° C. to 70° C.) and revert to the solid form upon cooling to room temperature.

In a particular set of embodiments the thermoplastic polymer matrix comprises polycaprolactone. Polycaprolactone (PCL) is one example of a biodegradable polymer having a low melting point that has been approved by United States Food and Drug Administration (USFDA). The melting point of PCL is related to its molecular weight, thus it is possible to select molecular weights that will have desired softening points as a result of having melting points within the desired range suitable for responding to the photo-thermal effect in vivo. Furthermore, the biodegradation rate of poly(caprolactone) is generally slow, which can be desirable for a long-term drug delivery application.

In one set of embodiment, the thermoplastic polymer matrix comprises polycaprolactone having a molecular weight (Mₙ) in a range of from about 1000 g/mol to 43,000 g/mol, or from about 2000 g/mol to 10,000 g/mol.

Polycaprolactone having a molecular weight (Mₙ) of about 2000 g/mol exhibits a melting temperature (Tm) of approximately 53° C., while polycaprolactone having a molecular weight of about 10,000 g/mol exhibits a melting temperature (Tm) of approximately 64° C. as determined by differential scanning calorimetry (DSC). Based on these melting temperatures, it is anticipated that the PCL would have a softening point in the range of from about 30° C. to 70° C. and begin to soften in the polymer composite in the desired temperature range.

In some embodiments, the thermoplastic polymer matrix may comprise a mixture of two or more polyesters. The thermoplastic polymer matrix may comprise a mixture of polyesters of different composition and/or different molecular weight.

In one set of embodiments, the thermoplastic polymer matrix comprises a polyester mixture comprising at least two polyester polymers of different molecular weight. For example, the thermoplastic polymer matrix may comprise a mixture of polycaprolactone of low molecular weight and polycaprolactone of high molecular weight. In one embodiment, the thermoplastic polymer matrix comprises a polyester mixture comprising polycaprolactone of molecular weight 2000 g·mol⁻¹ and poly(caprolactone) of molecular weight 10,000 g·mol⁻¹. Polycaprolactone of different molecular weights may exhibit different thermal or melting behaviours. The use of polymers of different molecular weight may therefore afford the ability to tune the overall thermoresponsive or softening characteristics of the thermoplastic polymer matrix.

The high molecular weight polyester and low molecular weight polyester may be combined in any suitable ratio. In one set of embodiments, the thermoplastic polymer matrix comprises a high molecular weight polyester and low molecular weight polyester, where the molar ratio of high molecular weight polyester to low molecular weight polyester is in a range selected from 5:95, 10:90, 20:80, 25:75, 50:50; 75:25; 80:20; 90:10 and 95:5. The choice of ratio may depend upon the molecular weight of the agent contained in the thermoplastic polymer matrix and its interaction with the polymer matrix. For example to slow down the release of a low molecular weight agent one might use a high proportion of a high molecular weight polymer but for a high molecular weight agent one would select a lower molecular weight polymer to allow faster drug release, depending on how much of the agent is required to elicit a desired response.

In another set of embodiments, the thermoplastic polymer matrix comprises a neat polyamide. The neat polyamide may be selected from the group consisting of poly(caprolactam) (Nylon 6), poly(hexamethylene adipamide) (Nylon 6,6), poly(hexamethylene dodecanamide) (Nylon 6,12), poly(ω-dodecanamide) (Nylon 12), and mixtures thereof.

In another set of embodiments, the thermoplastic polymer matrix comprises a neat polyether. Exemplary polyethers may be selected from the group consisting of poly(ethylene oxide), poly(propylene oxide), and their block co-polymers such as poloxamers (Pluronic®).

Exemplary polyamide and polyethers as described herein can exhibit a thermal phase transition (e.g. melting temperature) and thus a softening temperature (i.e. softening point) within the specified range of 30° C. to 70° C., depending on their molecular weight.

In another set of embodiments, the thermoplastic polymer matrix comprises neat poly(vinyl pyrrolidone) and/or a polyoxazoline, such as poly(ethyl oxazoline). Neat poly(vinyl pyrrolidone) and polyoxazolines can exhibit a thermal phase transition (e.g. glass transition temperature within the specified range of 30° C. to 70° C.), depending on the molecular weight of the polymer.

The polymer composite of the present invention also comprises an agent. The agent is dispersed within the thermoplastic polymer matrix of the composite. Release of the agent is modulated through the softening of the thermoplastic polymer matrix.

The polymer composite may comprise a variety of different agents and the present invention is not limited to specific types or forms of agent. A skilled person would appreciate that the type of agent contained in the thermoplastic polymer matrix of the composite will depend on the desired application in which the polymer composite is to be used. For example, the polymer composite may comprise agents such as dyes or compounds used as environmental sensors that may detect or complex with environmental pollutants.

In some embodiments, the polymer composite can be utilized for controlled release of active agent in biomedical (e.g. drug, hormone, gene, vitamin or cytokines) or agricultural applications (e.g. fertilizer, pesticide, herbicide or insecticide).

In particular embodiments, the polymer composite is for drug delivery applications and the agent contained in the thermoplastic polymer matrix is a drug.

As used herein, the term “drug’ relates to a substance used for the prevention, diagnosis, alleviation, treatment or cure of a disease or disorder in a subject. The drug is generally a chemical or biological substance, and may be prophylactic, diagnostic or therapeutic substance.

In one set of embodiments, the polymer composite comprises at least one drug. The drug may be selected from the group consisting of therapeutic agents, diagnostic agents, prophylactic agents, and combinations thereof.

In one set of embodiments, the drug may be selected from the group consisting of biologically active macromolecules (such as proteins or peptides), small molecules (i.e. having a molecular weight of no more than about 1000 Da), organometallic compounds, nucleic acids (e.g., DNA, RNA, RNAi, etc.), isotopically labeled chemical compounds, and combinations thereof.

Drugs that may be delivered by the polymer composite of the invention may further be hydrophilic or hydrophobic drugs.

In one embodiment, the drug is an organic compound with pharmaceutical activity, such as, for instance, a clinically used drug. Examples of drugs include but are not limited to antibiotics, antimicrobial agents, anti-viral agents, anaesthetics, steroidal agents, anti inflammatory agents, anti-neoplastic agents, antigens, vaccines, antibodies, growth factors, decongestants, antihypertensives, sedatives, birth control agents, progestational agents, anti-cholinergics, analgesics, anti-depressants, anti-psychotics, β-adrenergic blocking agents, diuretics, cardiovascular active agents, vasoactive agents, non-steroidal anti-inflammatory agents, nutritional agents, prostaglandin, etc. The drug may be used to treat a condition, such as cancer (e.g., as a chemotherapeutic agent) or a chronic disease (e.g., epilepsy, a neurodegenerative disease, a cardiovascular disease, an autoimmune disease, diabetes, etc.), etc.

In one form, the polymer composite comprises an anticancer agent. Examples of anti-cancer agents include, without limitation, methotrexate, trimetrexate, adriamycin, taxotere, doxorubicin, 5-fluorouracil, vincristine, vinblastine, pamidronate disodium, anastrozole, exemestane, cyclophosphamide, epirubicin, toremifene, letrozole, trastuzumab, megestrol, tamoxifen, paclitaxel, docetaxel, capecitabine, goserelin acetate, etc.

In one form, the polymer composite comprises an antimicrobial or antibiotic agent. Examples of antimicrobial or antibiotic agents include, without limitation, amoxicillin, chloramphenicol, ciprofloxacin, gentamycin, oxytetracycline, streptomycin, lysozyme, dexamethasone, levofloxacin, temafloxacin, cefoxitin, vancomycin, etc.

In one form, the polymer composite comprises an anti-inflammatory agent, such as a corticosteroid. Examples of corticosteroids include, without limitation, bethamethasone, prednisone, prednisolone triamcinolone, methylprednisolone, dexamethasone, hydrocortisone, cortisone, ethamethasoneb, and fludrocortisone.

In one form, the polymer composite comprises biologically active macromolecules. Examples of biologically active macromolecules include, without limitation, polypeptides and aptamers (including proteins such as enzymes, antibodies, and their fragments such as erythropoietin, follistatin, oxytocins, vasopressin, adrenocorticotropic hormone, epidermal growth factor, platelet-derived growth factor (PDGF), prolactin, luliberin, lutenizing hormone releasing hormone (LHRH), growth hormone, growth hormone releasing factor, insulin, somatostatin, glucagon, interleukin-2 (IL-2), interferon-α, -β, -γ, gastrin, uragastrone, secretin, calcitonin, endorphins, angiotensins, thyrotropin releasing hormone (TRH), tumor necrosis factor (TNF), macrophage-colony stimulating factor (M-CSF), heparinise, bone morphogenic protein (BMP), hANP, glucagon-like peptide (GLP-1), interleukin-11 (IL-11), bacitracins, polymyxins, colistins, tyrocidine, gramicidins, etc.).

In one form, the polymer composite comprises a molecular/biomacromolecular antagonist to different receptors. Such antagonists include, without limitation, vascular endothelial growth factor (VEGF), tumour necrosis factor (TNF), insulin, complement component C3, C5, amyloid-β, sphingosine-1-phosphate, factor D, IL-2 receptor subunit-α, and cyclooxygenase.

In one form, the polymer composite comprises a vascular endothelial growth factor (VEGF) antagonist. A VEGF antagonist is a molecule that is capable of neutralizing, blocking, inhibiting, abrogating, reducing or interfering with activities of a native sequence VEGF, including its binding to one or more VEGF receptors.

Drugs belonging to the class of VEGF antagonists (also known as VEGF inhibitors) can be used to treat degenerative eye conditions, such as wet age-related macular degeneration.

Examples of VEGF antagonists include, without limitation, proteins such as aflibercept, bevacizumab, conbercept, pegaptanib and ranibzumab.

The polymer composite may comprise a mixture of agents in some cases, e.g., a mixture of drugs. For instance, one or more drug species may be present in a single polymer composite. Drug mixtures may be desirable for combination therapy. For example, a VEGF inhibitor (bevacizumab, ranibizumab, or aflibercept) may be combined with an anti-inflammatory agent (e.g. triamcinolone derivatives) in order to reduce the inflammation and blood vessel growth for age-related macular degeneration (AMD) or diabetic retinopathy therapy.

The polymer composite of the present invention can be prepared in any suitable manner. In one embodiment, the polymer composite may be prepared by heating the polymer selected for formation of the thermoplastic polymer matrix (either as a neat polymer or as a polymer in solution) above its thermal transition temperature (e.g. glass transition temperature or melting temperature) so as to convert the polymer to a molten or liquid state. A polymer in solution would generally dissolve in the solvent at elevated temperature. The solvent may be an aqueous liquid, preferably water.

The agent to be dispersed within the thermoplastic polymer matrix may then be added and mixed with the molten polymer or polymer solution. Subsequently, a plurality of non-carboxylic acid stabilised photo-thermal particles may then be added and mixed together with the molten polymer or polymer solution containing the agent. The resulting liquid may then be rapidly cooled to a temperature that is below the glass transition temperature or melting temperature of the polymer, resulting in solidification of the polymer and encapsulation of the stabilised photo-thermal particles and agent within the solidified polymer.

In one aspect, the present invention provides a process for preparing a polymer composite of one or more embodiments described herein, the process comprising the steps of forming a liquid polymer mixture comprising at least one polymer, at least one agent and a plurality of non-carboxylic acid stabilised photo-thermal particles, and solidifying the liquid polymer mixture to form the polymer composite.

The polymer contained in the liquid polymer mixture forms part of the thermoplastic polymer matrix of the polymer composite.

Solidification of the liquid polymer mixture may occur by components in the liquid mixture participating in covalent or non-covalent intermolecular bonding interactions.

In one embodiment, solidification of the liquid polymer mixture may involve the step of crosslinking the polymer contained in the polymer mixture. Crosslinking may via covalent or non-covalent intermolecular interactions. For examples, chains of polymer in the liquid polymer mixture may crosslink with itself or with an additive, such as a crosslinking agent, that is optionally added to the liquid polymer mixture. Crosslinking may be facilitated by temperature or via chemical means. If desired, crosslinking may be facilitated by processes, such as curing processes that promote inter-molecular interactions and bond formation.

In one set of embodiments, the liquid polymer mixture further comprises a crosslinking agent and solidification of the liquid polymer mixture involves the step of covalently crosslinking the polymer and the crosslinking agent to form covalent bonds between chains of the polymer and the crosslinking agent. In one preference, the liquid polymer mixture is photo-catalytically cured. Photo-curing of the liquid polymer mixture may be achieved using a suitable source of light, such as UV light.

In one embodiment, solidification of the liquid polymer mixture involves photo-curing or photo-catalytically cross-linking of the liquid mixture as a bulk volume (i.e. in the form of a bulk liquid polymer mixture) to form a bulk polymer composite.

In some embodiments, solidification of the liquid polymer mixture may be achieved by cooling the liquid polymer mixture to a desired temperature. In one preference, the liquid polymer mixture is cooled to a temperature that is less then 30° C. For example, when agarose is used to prepare the polymer composite, a liquid polymer mixture comprising agarose solidifies when it is cooled.

In one embodiment, solidification of the liquid polymer mixture involves cooling the liquid polymer mixture as a bulk volume to form a bulk polymer composite.

In some embodiments, solidification of the liquid polymer mixture may be achieved by heating the liquid polymer mixture to a desired temperature. In one preference, the liquid polymer mixture is heated to a temperature that is about 30° C. or above. For example, when a poloxamer is used to prepare the polymer composite, a liquid polymer mixture comprising the poloxamer solidifies when it is heated up to approximately physiological temperature (37° C.).

In one embodiment, solidification of the liquid polymer mixture involves heating the liquid polymer mixture as a bulk volume to form a bulk polymer composite.

Solidification of the liquid polymer mixture results in encapsulation of the agent and stabilised photo-thermal particles within the polymer that forms part of the thermoplastic polymer matrix of the polymer composite. Chains of polymer in the liquid mixture may undergo physical entanglement or cross-linking during solidification to form the polymer composite.

In some embodiments, the polymer in the liquid polymer mixture may undergo covalent or non-covalent intermolecular interactions with the non-carboxylic acid stabiliser that stabilises the photo-thermal particles, leading to cross-linking between the photo-thermal particles and the resultant polymer matrix. The intermolecular interaction between the polymer and the stabilised photo-thermal particles may aid the dispersion of the photo-thermal particles in the polymer matrix.

In some embodiments, solidification of the liquid polymer mixture may involve dispersion of the liquid mixture as a dispersed phase in a continuous phase. This can lead to the formation of a plurality of discrete polymer composite particles, such as polymer composite microparticles. The liquid polymer mixture may be added dropwise into a continuous phase to be dispersed in the continuous phase. In some embodiments, the liquid polymer mixture may be introduced as a stream, which is then dispersed in the continuous phase under shear, which can produce droplets of the polymer mixture in the continuous phase. The continuous phase may be maintained at a temperature that facilitates solidification of the dispersed liquid polymer mixture.

In embodiments where a liquid polymer mixture is dispersed in a continuous phase, it can be preferable for the liquid mixture to be an aqueous mixture and be dispersed in a continuous oil phase. This can allow polymer composite particles to be formed in a water-in-oil (W/O) emulsion.

In one embodiment of the process, formation of the liquid polymer mixture may comprise the steps of forming a liquid containing at least one polymer, and adding an agent and a plurality of non-carboxylic acid stabilised photo-thermal particles to the polymer-containing liquid. The agent and photo-thermal particles may be added simultaneously or sequentially to the polymer-containing liquid.

In some embodiments the polymer-containing liquid may be a polymer solution comprising at least one polymer dissolved or dispersed in a solvent. The solvent may be an organic solvent or an aqueous solvent. In one preference, the solvent is water. Examples of polymers suitable for forming the polymer composite of the invention are described herein. Mixtures of two or more polymers may be dissolved or dispersed in the solvent.

In other embodiments, the polymer-containing liquid may be a polymer melt comprising at least one molten polymer in liquid form. In some embodiments, the polymer-containing liquid may comprise two or more molten polymers in liquid form.

In other embodiments, formation of the liquid polymer mixture may comprise the steps of forming a liquid containing at least one selected from an agent and a plurality of non-carboxylic acid stabilised photo-thermal particles and adding at least one polymer to the liquid. If not already present, the other selected from the agent and the plurality of photo-thermal particles can also be added to the liquid simultaneously or sequentially with the polymer.

In yet other embodiments, formation of the liquid polymer mixture may involve the addition, either simultaneously or sequentially, of a polymer, an agent and a plurality of non-carboxylic acid stabilised photo-thermal particles to a solvent to form a liquid polymer solution.

The preparation of the polymer composite can be achieved by a batch process or by a continuous process. A continuous process may be a flow process, such as a microfluidic process. One example of a microfluidic process is described in ACS Chem. Biol., 2011, 6, 260-266. A microfluidic process may be advantageous for the preparation of the polymer composite of the invention in microparticle form, for example, for the preparation of coated hydrogel microparticles.

An advantage of the process described herein is the ease of fabrication of the polymer composite as a drug depot, whereby the drug can be incorporated into the polymer composite in a ‘one-pot’ fabrication method. In other drug delivery systems, subsequent loading (or post-loading) of the drug into the polymer composite can be limited by the size of the drug and the physicochemical properties of the polymer composite, such as storage modulus, porosity, charge and swelling ability. In comparison to these other drug delivery systems, the process described herein is able to pre-load large molecular weight drugs, such as proteins, into the polymer composite due to the ‘one-pot’ fabrication process.

The polymer composite of the invention may be fabricated into a variety of shapes, including two-dimensional and three-dimensional shapes. Examples of shapes include, but are not limited to, rods (such as cylindrical rods), particles (such as spherical particles) and films.

In some instances it can be desirable for the polymer composite to be injectable. This may be advantageous when the polymer composite of the invention is used for drug delivery applications where the polymer composite can be administered to a biological environment or a subject body by minimally invasive injection, instead of invasive surgical implantation. In such circumstances it can be desirable to fabricate the polymer composite into a shape that facilitates administration of the composite via injection through the lumen of a needle.

The term “injectable” as used herein refers to the ability to be injected through a surgical needle or catheter for administration subcutaneously, sublingually, buccally, intraocularly, topically or intramuscularly to a subject. It specifically excludes intravenous administration due to a risk of blockages in small veins or arteries caused by the polymer composite.

In one form, the polymer composite is fabricated into the shape of a cylindrical rod or particle having at least one dimension in the micron range.

In one set of embodiments, the polymer composite is fabricated into cylindrical rods. Preferably, the cylindrical rods have a diameter of between 1 μm and 1000 μm, or between 10 μm and 200 μm.

In one set of embodiments, the polymer composite is fabricated into spherical particles. The particles may be microparticles having at least one dimension in the micron range. Preferably, the microparticles have a diameter of between 1 μm and 1000 μm, or between 10 μm and 200 μm.

Polymer composite in the form of microparticles may be fabricated to be of a size and shape that aids in its injectability and its use in tissue implantation. Typical needle gauge used for subcutaneous injection is 25 G (inner diameter: 260 μm) and intravitreal injection is 30 G (inner diameter: 159 μm). The microparticles can be fabricated to suit injection by needles of such gauges.

In some embodiments, it is preferred that the polymer composite of the invention is not in the form of nanoparticles as nanoparticles may be at risk of being taken up by cells.

The fabrication of the polymer composite into particular shapes might increase the surface area of the composite. An increase in surface area may result in high release rates (e.g. burst release) for the agent contained in the composite. In some optional embodiments, it may be desirable to further control the release rate of the agent by containing the polymer composite with an additional polymer. This may help to minimise the burst release of the loaded agent, enhance the on-off release ratio, and prolong the sustained release of the agent from the polymer composite.

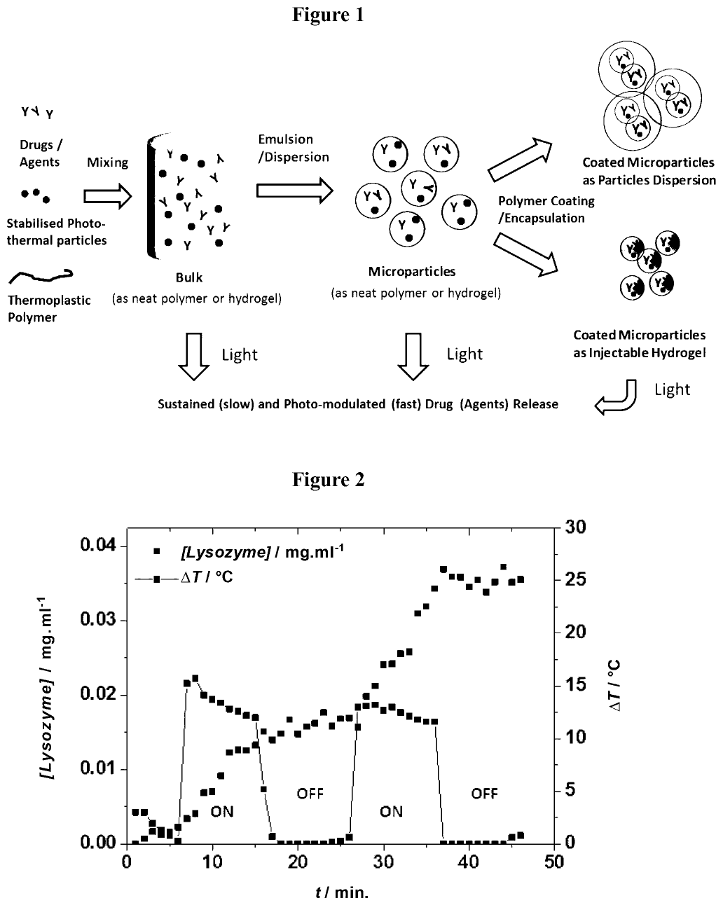

Containment of the polymer composite may be in one of two ways; by coating the polymer composite with an additional polymer or by enclosing the polymer composite within an additional polymer in the form of a bulk polymer film, as shown in FIG. 1.

Containment of the polymer composite by either a polymer coating or bulk polymer may be particularly suitable where the polymer composite is in the form of particles, preferably spherical particles, more preferably microparticles.

In one preference, a polymer composite contained within a polymer coating or bulk polymer remains implantable and/or injectable.

In some embodiments, a coated polymer composite is in the form of coated microparticles, where the surface of each microparticle is substantially (preferably entirely) coated by an additional polymer.

In one form, when the polymer composite is in the form of microparticles, containment of the polymer composite in a bulk polymer film means that the polymer composite microparticles are embedded within a bulk polymer (such as a hydrogel). The bulk polymer may be of the same composition or a different composition as the thermoplastic polymer matrix used in the polymer composite. The shape of the bulk polymer containing the polymer composite microparticles is such that the material is injectable.

In another aspect, the present invention provides an article or device comprising the polymer composite of one or more embodiments of the invention. The polymer composite may form part of or be formed into an article or device, or be applied as a coating on an article or device. Such devices include a transdermal delivery device or article, such as a patch, that is designed to contact the surface of the skin or the cornea of a subject.

In some embodiments, the polymer composite forms part of, or is formed into, a medical device. The medical device may be designed to be implanted in a subject. By being “implanted” is meant that the device is totally or partly introduced medically into a subject's body and which is intended to remain there after the procedure.

The polymer composite and articles or devices comprising the polymer composite may be administered to an individual via any route known in the art. These include, but are not limited to, oral, ocular, sublingual, nasal, intradermal, subcutaneous, subconjunctival, intravitreal, intramuscular, rectal, vaginal, intravenous, intraarterial, and inhalational administration.

The composite of the invention may be used as a biomaterial in a range of contexts, for example in materials, products or substances which are for use in contact with biological samples, tissues, fluids, cells, cell components, etc either ex vivo, in vivo or in vitro.

A medical device comprising the polymer composite may be implanted in any suitable location in a subject an area where delivery of a drug is needed, or in an area providing ready access to the bloodstream or to the brain, depending on the application. For instance, the device may be implanted subcutaneously, on or proximate a nerve or an organ, etc.

In one set of embodiments, the polymer composite of the invention may be formed into an article or device that is suitable for administering at least one drug to an eye of a subject.

In one form, the polymer composite is formed into an article or device that is suitable for implantation into an eye of a subject.

The polymer composite may be in the form a solid article, a film, a gel, or other form suitable for implantation in an eye of a subject. For instance, the polymer composite may be in a form that is suitable for injection into an eye of a subject.

In one set of embodiments, the polymer composite is in a form that may be injected through the lumen of a needle for implantation in an eye of a subject. For example, the polymer composite may be formed into spherical microparticles, which may be suspended in a solution for injection in an eye of a subject.

In some embodiments, a polymer composition that is capable of forming a polymer composite of the invention in situ may be injected through a needle to a desired site of administration. The polymer composition may be in liquid form for injection and convert into a solid or semi-solid form in situ after administration. For example, a liquid polymer composition may be administered by injection to a body site and when the temperature is raised above physiological temperature (e.g. temperature ≥40° C.) or cooled to below physiological temperature (e.g. temperature ≤30° C.), the liquid composition may then be converted into a solid or semi-solid (gel) polymer composite.

In another aspect, the present invention provides an ocular implant comprising a polymer-composite of any one of the embodiments described herein. Preferably, the ocular implant is for the treatment or prophylaxis of an ocular disease or disorder. Examples of ocular diseases or disorders include age-related macular degeneration (AMD), uveitis, diabetic retinopathy, macular oedema, ocular uveitis, endophthalmitis and glaucoma.

An ocular implant comprising the polymer composite may be in the form of a particle, such as a microparticle. A microparticle would have at least one dimension in the micron range. In some embodiments, an ocular implant in the form of a microparticle has a diameter in the micron range, such as a diameter in a range of from about 1 to 100 μm, from about 10 to 90 μm, from about 20 to 80 μm and from about 30 to 70 μm.

The polymer composite of the present invention has therefore been envisioned for ophthalmic applications to minimize the frequency of intraocular injection of a drug, such as a therapeutic antibody, which can be released from the polymer composite in a photo modulated manner.

In another aspect, the present invention provides an implantable article comprising a polymer composite of any one of the embodiments described herein and a thermoplastic polymer encapsulating the polymer composite. The implantable article may be an injectable article.

In one form, the implantable article comprises the polymer composite in the form of particles, preferably spherical particles, more preferably microparticles. The microparticles are contained within the thermoplastic polymer.

Thus in another aspect, the present invention provides an implantable article comprising one or more microparticles comprising the polymer composite of any one of the embodiments described herein and a thermoplastic polymer containing the microparticles.

Containment of the microparticles may occur by way of a surface coating of thermoplastic polymer that entirely surrounds each microparticle or alternatively, by way of a bulk thermoplastic polymer that encloses the microparticles.

In one form of the implantable article, the microparticles may be surface coated with a thermoplastic polymer, which surrounds each microparticle.

In another form of the implantable article, the microparticles may be dispersed and enclosed within a bulk thermoplastic polymer film.

The thermoplastic polymer coating or enclosing the polymer composite is preferably capable of softening in response to an increase in temperature. Accordingly, upon heating of the polymer composite, the polymer coating or bulk material coating the composite also softens in response to the heat. The polymer coating or bulk polymer film therefore also exhibits a thermomechanical softening property and as such, may also be regarded as a thermoplastic polymer. A thermoplastic polymer forming the polymer coating or bulk polymer film may be a neat polymer or a hydrogel polymer with a softening temperature (i.e. softening point) in a range of from about 30° C. to 70° C., as determined by rheometer.

When the polymer composite of the invention is coated or enclosed by a thermoplastic polymer, it is also preferred that the softening point of the thermoplastic polymer forming the polymer coating or bulk polymer is lower than that exhibited by the thermoplastic polymer matrix of the polymer composite. This can help to ensure that the polymer coating or bulk polymer softens at a lower temperature than that of the thermoplastic polymer matrix and thus does not detrimentally impede the diffusion or release of the agent from the matrix to the surrounding environment, such as the biological environment. It can also ensure that any thermal effects that may arise during the process used to coat or enclose the pre-fabricated polymer composite can be minimised.

The thermoplastic polymer employed to coat or enclose the polymer composite may comprise a suitable neat polymer or hydrogel, and examples of such neat polymers and hydrogels are described herein before.

In one form, the implantable article comprises a plurality of microparticles comprising a polymer composite of any one of the embodiments described herein and a thermoplastic hydrogel coating or enclosing the microparticles. The hydrogel coating preferably comprises a polymer selected from the group consisting of a polysaccharide, a polypeptide or a poloxamer, in the polymer phase. More preferably, the hydrogel comprises agarose, gelatine or a poloxamer in the polymer phase.

In one form, the thermoplastic polymer that coats or encloses the polymer composite microparticles may be of the same type or of a different type of polymer as the thermoplastic polymer matrix of the polymer composite.

In some embodiments, the thermoplastic polymer that is present in the matrix of the polymer composite and in the polymer coating are of the same type of polymer.

In one example, when the thermoplastic polymer matrix of the polymer composite comprises neat polycaprolactone, the thermoplastic polymer that coats or encloses the polymer composite may also comprise neat polycaprolactone. However, the polycaprolactone used as the coating or enclosing polymer may be of lower molecular weight than that used in the thermoplastic polymer matrix of the composite, as a lower molecular weight polycaprolactone can have a lower softening point and thus be able to soften at a lower temperature.

In another example, when the thermoplastic polymer matrix of the polymer composite comprises a hydrogel, such as an agarose hydrogel, the thermoplastic polymer that coats or encloses the polymer composite may also comprise an agarose hydrogel. In this instance, the agarose hydrogel used as the coating or enclosing polymer may have a lower agarose (solids) content, or alternatively, the agarose hydrogel may comprise a lower melting agarose, to ensure that the coating or enclosing polymer softens at a lower temperature.

Implantable articles may be implanted directly or formulated and then implanted. If an article is injected, the articles may also be formulated or injected alone. Injectable preparations, for example, sterile injectable aqueous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent. Among the acceptable vehicles and solvents that may be employed are water, Ringer's solution, U.S.P. and isotonic sodium chloride solution. In addition, sterile, fixed oils may be employed as a solvent or suspending medium. For this purpose any bland fixed oil can be employed including synthetic mono- or diglycerides. In addition, fatty acids such as oleic acid can be used in the preparation of injectables. In some embodiments, the articles may be suspended in a carrier fluid comprising 1% (w/v) sodium carboxymethyl cellulose and 0.1% (v/v) Tween 80.

The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

If the articles are delivered to a subject by alternative routes, they may be prepared in formulations suitable or oral, rectal, vaginal, nasal, subcutaneous, or pulmonary delivery.

The polymer composite of the invention provides for controlled delivery of an agent, such as a drug. By “controlled delivery” is meant that the agent or drug is released from the polymer composite in a pre-determined or controlled manner. Generally, release of the agent or drug occurs when the polymer composite is exposed to light. As a result, a quantity or dose of the agent or drug can be released from the polymer composite when it is needed to achieve a desired effect.

The polymer composite of the invention can also provide for sustained delivery of an agent, such as a drug. By “sustained delivery” is meant that the agent or drug is released from the polymer composite over a period of time, for example over a period of 10 or more minutes, 30 or more minutes, 60 or more minutes, 2 or more hours, 4 or more hours, 12 or more hours, 24 or more hours, 2 or more days, 5 or more days, 10 or more days, 30 or more days, 2 or more months, 4 or more months or over 6 or more months.

When the polymer composite is in use, the stabilised photo-thermal particles absorb photo-energy and convert that photo-energy into thermal energy. The thermal energy is emitted as heat to the thermoplastic polymer matrix. The thermoplastic polymer matrix in turn softens due to an increase in temperature arising from the heat, resulting in the agent contained in the matrix being released to the surrounding environment.

When the source of photo-energy (i.e. light source) is removed, heating of the thermoplastic polymer matrix via the photo-thermal effect ceases. The thermoplastic polymer matrix therefore cools, resulting in re-solidification of the matrix. The solification of the thermoplastic polymer matrix slows or stops the release of the agent from the thermoplastic polymer matrix.

The polymer composite of the present invention allows release of an agent that is dispersed in the thermoplastic polymer matrix to be switched on and off when desired by either exposing the polymer composite to light (‘on’ mode) or removing the light source (‘off’ mode).

The release of an agent, such a drug, from the polymer composite can be modulated by varying a number of parameters, including the concentration, size and/or shape of the photo-thermal particles in the polymer composite, the amount and type of polymer present in the thermoplastic polymer matrix of the composite, and the intensity, wavelength and/or frequency of radiation applied of the polymer composite. Adjustments of these parameters enable the release profile of the agent to be controlled. Optional coating of the polymer composite within a coating polymer as described herein may also provide for additional control over the delivery of the agent. The present invention therefore provides a versatile polymer composite that can be provide for modulated delivery of an agent, which can be tuned to accommodate specific conditions or to target specific diseases.

The polymer composite of the invention can provide for sustained delivery of an agent by slowly releasing the agent over a period of time. Release of the agent from the polymer composite may then be accelerated when desired by exposing the composite to light. For example, the polymer composite may allow a drug to be released slowly over time for the treatment of a disorder or disease and when required or desired, a larger dose of the drug may be supplied by exposing the polymer composite to light.

The invention will now be described with reference to the following examples. However, it is to be understood that the examples are provided by way of illustration of the invention and that they are in no way limiting to the scope of the invention.

### EXAMPLES

Chemicals and Materials:

Gold chloride trihydrate (HAuCl₄; 99.9%) was purchased from Sigma-Aldrich (Australia) and tri-sodium citrate (Na₃C₆H₅O₇.2H₂O) was purchased from Ajax Finechem. Monomers [2-(methacryloyloxy) ethyl] trimethyl ammonium chloride (METAC; 75%) and oligo(ethylene glycol) methyl ether methacrylate (OEGMA; Mₙ: 300 g/mol) were purchased from Sigma-Aldrich (Australia) and was purified by precipitation into acetone before use. 2,2′-Azobis-(isobutyronitrile) (AIBN) was purchased from Sigma-Aldrich (Australia) and was crystallized twice from ethanol prior to use and then stored at 4° C. RAFT agent 4-cyano-4-(thiobenzoylthio) pentanoic acid (CTA, 97%) was purchased from Strem Chemicals (Australia) and used as received. Acetone, methanol, ethanol, diethyl ether, dichloromethane were purchased from Merck. Lysozyme (lyophilized powder from chicken white egg, L6876), bovine serum albumin (BioXtra, A3311), and immunoglobulin G (lyophilized powder from human blood, G4386) were purchased from Sigma-Aldrich, Australia. 0.9% Sodium chloride was supplied by Baxter, and was used to solubilize lysozyme, bovine serum albumin, immunoglobulin G, and bevacizumab (Avastin®). Agarose (Nusieve GTG) and low gelling Agarose (Type VII-A) were purchased from Lonza (Australia). Poly(caprolactone)s with the molecular weight Mₙ: 2,000; 10,000; 25,000; and 43,000 g/mol were purchased from Sigma-Aldrich (Australia). Poloxamers (Pluronic® F127, F108, F87, F88, and F68) were purchased from Sigma-Aldrich (Australia) and received from BASF (Australia). Poly(vinyl alcohol) (Mₙ: 30,000-70,000 g/mol, 90% hydrolyzed), Span® 80, Tween® 80, and soya bean oil were purchased from Sigma-Aldrich (Australia), and used in the emulsion to generate microparticles dispersion. Avastin® or bevacizumab injection (Roche, solution of 100 mg in 4 mL 0.9% saline). Water was purified with a Millipore Milli-Q system. All other reagents were used without further purification.

Instrumentation:

NMR analysis of RAFT-synthesized poly(methacryloxyethyl trimethylammonium chloride) and poly(oligoethylene glycol methyl ether methacrylate) was performed with a Bruker Av 400 NMR spectrometer, before and after aminolysis. ¹H NMR spectra were recorded in deuterium water (D₂O). For aqueous GPC analysis of poly(methacryloxyethyl trimethylammonium chloride) and poly(oligoethylene glycol methyl ether methacrylate), three Eprogen CATSEC columns 100, 300, and 1000 (5 micron; 250×4.6 mm) and Eprogen CATSEC guard column 100 (7 microns; 250×4.6 mm) were used, using water (containing 1 v/v % acetic acid and 0.1 M Na₂SO₄) at 0.3 ml/min as the eluent on a system with a differential refractive index detector calibrated with linear poly(ethylene oxide) standards.

The concentration of gold nanoparticles was measured using a Cary 50 Bio UV-visible spectrophotometer (Varian Co., USA) performed at room temperature. Particle size distribution and zeta potential (ζ) of gold nanoparticles (AuNPs) were measured at 25° C. in standard disposable cuvettes using a Zeta sizer-Nano instrument (Malvern, UK) running DLS software and operating a 4 mW He—Ne laser at 633 nm (scattered light was performed at an angle of 173°). Neat and polymer-stabilised gold nanoparticles (AuNPs) in water were filtered through Millipore nylon filters (pore size 0.45 μm) to eliminate dust and large contaminants prior to analysis. The temperature was allowed to equilibrate for 2 minutes. The results were determined on an average of five measurements. The agarose hydrogel was modified to microbeads through w/o emulsion, in order to measure its zeta potential (ζ).

ATR-FTIR analysis was performed with a Thermo Scientific Nicolet 6700 FT-IR. The samples were pre-dried in vacuum overnight prior to measurement (each 320 scans).

The morphology and size of polymer-stabilised AuNPs were examined by Transmission Electron Microscopy (TEM). The sample on a copper grid was imaged using a Tecnai 12 transmission electron microscope (FEI, Eindhoven, The Netherlands) at an operating voltage of 120 kV, equipped with a MegaView III CCD camera and analysis imaging software (Olympus Soft Imaging Solutions).

Light source (Omnicure Series 2000, USA) with adjustable power/light intensity was used with 400-500 nm filter to generate blue light for drug release experiment. UV-Vis detector probe (C Technologies, Inc., USA) was used to online monitor the concentration of released drug. Dual thermocouple (Tenma, USA) was used to online monitor the temperature of AuNPs/hydrogel system and the PBS solution.

Rheological study was conducted using an ARES rheometer (TA Instruments, USA) connected to a light source (400-500 nm, 0.5 W) via a fibre optic. The thermoplastic polymer matrix sample was loaded in the center of two parallel plates of 20 mm diameter at room temperature (25° C.). The gap between the two plates was set at 0.3 mm. The storage shear modulus (G′), loss shear modulus (G″) and viscosity (η*) were measured as a function of time at a constant frequency of 10 rad/s and a strain of 1.0%. Depending on the type of thermoplastic polymer matrix, the sample (100 μl) was loaded as solution for hydrogel or as melt for neat dry polymer film at 45-65° C., and set for about 35 minutes at 25° C. until the G′ reached saturation, followed by the blue light exposure for 10 minutes.

The melting temperature (Tₘ) and glass transition temperature (Tg) of polymer matrix (in the form of dry neat polymer film, hydrogel, or microparticles) were determined by differential scanning calorimetry (DSC) measurements (Mettler Toledo, Switzerland) under N₂ atmosphere.

The dynamic mechanical thermal analysis (DMTA) of the thermoplastic polymer matrix film was conducted using an ARE rheometer (TA Instruments, USA) with a pelltier. The sample was loaded in the center of two parallel plates of 20 mm diameter. The gap between the two plates was set at 0.3 mm. The storage shear modulus (G′), loss shear modulus (G″) and viscosity (η*) were measured as a function of time at a constant frequency of 1 rad/s and a strain of 1.0%. Depending on the type of thermoplastic polymer matrix, the sample (100 μl) was loaded as solution for hydrogel or as melt for neat dry polymer film, and set for about 35 minutes at 20° C. until the G′ reached saturation. The temperature was increased and decreased in two cycles at 1° C./mins rate, and kept isotherm at maximum (20° C.) and minimum (70° C.) for at least 3 minutes. The onset of the reduction in viscosity (η*) and storage shear modulus (G′) is determined as the softening temperature according to ASTM E1640.

The microparticles were observed on Optical microscope (Nikon, Japan). The scanning electron microscopy (SEM) image was observed using Camscan SEM and energy dispersive spectroscopy (EDS) was measured on X-ray analysis (Aztec, Oxford).

General Procedures:

Preparation of Polymer Stabilised Gold Nanoparticles (AuNPs)

The synthesis of poly(methacryloxyethyl trimethylammonium chloride) and the modification of gold nanoparticles using this polymer were described by Astolfo et al. in Nanomed. Nanotech. Biol. Med. 2014, 10, 1821-1828. In summary, neat gold nanoparticles (60-70 nm diameter) were prepared by the reduction of HAuCl₄ with sodium citrate in aqueous solution. The nanoparticles were stabilised by adding 0.3 mg/mL of thiol-terminated poly(methacryloxyethyl trimethylammonium chloride) to the solution and mixed for 30 minutes. The solution was centrifuged at 3200 g for 50 minutes. The supernatant was removed and the concentrated solution was used.

The same procedure was used to prepare gold nanoparticles stabilized with poly(oligoethylene glycol methyl ether methacrylate) (P(OEGMA)).

Release of Agent from Polymer Composite Under Blue Light Exposure

A polymer composite loaded with an agent was pre-heated in the water bath at 37° C. to mimic the body temperature before light exposure. After about 5-10 mins, the sample was exposed with blue light (400-500 nm) from a distance of ˜0.5 cm. The light intensity was pre-set, for example, 1.44 W power to generate about 508 mW/cm² light intensity on the surface of the AuNPs/hydrogel. The exposure time was set to 10 minutes for every blue light exposure (“ON”). After about 10 minutes of “OFF” interval, the blue light exposure was repeated (2 times for total 50 minutes, or 3 times for total 70 minutes). The temperatures of polymer composite and the surrounding PBS solution were monitored online by double thermocouple over period of time. The concentration of released agent was monitored online by using UV-Vis detector probe over period of time. UV-Vis calibration was used to calculate the UV-Vis absorbance of the released agent to their concentration. The agent release rate was determined by the slope of the agent release profile. Polymer composites loaded with an agent but without photo-thermal particles (AuNPs) were used as comparison for agent release rate and temperature increase.

Bioactivity Test for Released Lysozyme

The bioactivity of the protein, lysozyme, was measured by determining the lysis rate of Micrococcus lysodeikticus mediated hydrolytically by lysozyme in accordance with reported procedure (A. Ghaderi, J. Carlfors, Pharm. Res. 1997, 14, 1556-1562).

Bioactivity Test for Released Bevacizumab (Avastin®)

A literature method was employed to determine the bioactivity of released Avastin®. Based on its specific binding activity to human VEGF-165 (165 isoform of VEGF-A), the concentration of bevacizumab (Avastin®) was measured with an enzyme-linked immunosorbent assay (ELISA) method. D. Ternant, N. Ceze, T. Lecomte, D. Degenne, A.-C. Duveau, H. Watier, E. Dorval, G. Paintaud, Ther. Drug Monit. 2010, 32, 647-652.

Structural Stability Test for the Released Bevacizumab (Avastin®)

The structural ability of released antibodies and other proteins are assessed by gel filtration chromatography. Samples of released antibody and other proteins from the polymer composites were manually injected into the ÄktaPurifier (GE) for separation on a Superdex 200 GL 10/30 gel filtration column (GE) using PBS as eluent, which was set to run at 0.5 ml/min flowrate. Chromatogram profiles of all samples, measured at 280 nm, were analysed using the UNICORN v5.11 software. The approximate molecular weights were assessed by separating an aliquot of gel filtration standards (Bio-Rad) on the same column using parameters described above. Aggregation and degradation products can be characterized by the emergence of peaks with higher and lower molecular weights than the sample, respectively.

### Experiment 1

Polymer Composite with Agarose Hydrogel Polymer Matrix

A polymer composite comprising, polymer-stabilized gold nanoparticles (AuNPs) and a polymer matrix of 2% or 4% agarose was fabricated by solubilizing 20 mg or 40 mg agarose in 700 mg Milli-Q water at 75° C. When the temperature was reduced to 55° C., 100 μL of 1 mg/mL poly(methacryloxyethyl trimethylammonium chloride)-stabilised AuNPs were added, followed by vortex mixing, and incubation at 55° C. for 30 minutes. Successful incorporation of the AuNPs was confirmed by ATR-FTIR. When the temperature was further reduced to 45° C., the agent was added followed by vortex mixing. In some cases this was 200 μL of 25 mg/mL Lysozyme in 0.9% saline. After incubation for 5 minutes, the mixture was rapidly chilled at 15° C. to solidify the protein-loaded AuNPs/hydrogel polymer composite. Results are shown in Table 1.

T1

| Example | | | |
|---|---|---|---|
| No | Polymer Matrix | Stabilised Photo- | Agent |
| (%w/w) | (%w/w) | thermal particle | (%w/w) |
| 1 | Agarose hydrogel | P(METAC)-stabilised | Doxorubicin |
| | (4%) | AuNPs (0.01%) | (0.2%) |
| 2 | Agarose hydrogel | P(METAC)-stabilised | Triamcinolone |
| | (4%) | AuNPs (0.01%) | acetonide |
| | | | (0.25%) |
| 3 | Agarose hydrogel | P(METAC)-stabilised | Lysozyme |
| | (4%) | AuNPs (0.01%) | (0.25%) |
| 4 | Agarose hydrogel | P(METAC)-stabilised | Lysozyme |
| | (4%) | AuNPs (0.03%) | (0.25%) |
| 5 | Agarose hydrogel | P(METAC)-stabilised | Lysozyme |
| | (4%) | AuNPs (0.05%) | (2%) |
| 6 | Agarose hydrogel | P(METAC)-stabilised | Lysozyme |
| | (4%) | AuNPs (0.1%) | (2%) |
| 7 | Agarose hydrogel | P(METAC)-stabilised | Lysozyme |
| | (4%) | AuNPs (0.5%) | (2%) |
| 8 | Agarose hydrogel | P(METAC)-stabilised | BSA (2%) |
| | (2%) | AuNPs (0.01%) | |
| 9 | Agarose hydrogel | P(METAC)-stabilised | BSA (2%) |
| | (4%) | AuNPs (0.01%) | |
| 10 | Agarose hydrogel | P(METAC)-stabilised | BSA (0.5%) |
| | (2%) | AuNPs (0.01%) | |
| 11 | Agarose hydrogel | P(METAC)-stabilised | IgG (0.5%) |
| | (4%) | AuNPs (0.01%) | |
| 12 | Agarose hydrogel | P(METAC)-stabilised | IgG (0.5%) |
| | (2%) | AuNPs (0.01%) | |
| 13 | Agarose hydrogel | P(METAC)-stabilised | IgG (0.5%) |
| | (4%) | AuNPs (0.05%) | |
| 14 | Agarose hydrogel | P(METAC)-stabilised | IgG (0.5%) |
| | (2%) | AuNPs (0.05%) | |
| 15 | Agarose hydrogel | P(METAC)-stabilised | IgG (0.25%) |
| | (4%) | AuNPs (0.01%) | |
| 16 | Agarose hydrogel | P(METAC)-stabilised | IgG (0.25%) |
| | (2%) | AuNPs (0.01%) | |
| 17 | Agarose hydrogel | P(METAC)-stabilised | IgG (0.25%) |
| | (4%) | AuNPs (0.05%) | |
| 18 | Agarose hydrogel | P(METAC)-stabilised | IgG (0.25%) |
| | (2%) | AuNPs (0.05%) | |
| 19 | Agarose hydrogel | P(METAC)-stabilised | Bevacizumab |
| | (2%) | AuNPs (0.01%) | (0.125%) |
| 20 | Agarose hydrogel | P(METAC)-stabilised | Bevacizumab |
| | (2%) | AuNPs (0.05%) | (0.125%) |
| 21 | Agarose hydrogel | P(METAC)-stabilised | Bevacizumab |
| | (4%) | AuNPs (0.05%) | (0.25%) |

P(METAC) = poly(methacryloxyethyl trimethylammonium chloride)
IgG = Immunoglobulin G;
BSA = bovine serum albumin.

### Experiment 2

Fabrication of Microparticles from Polymer Composite with Agarose Hydrogel Polymer Matrix

The sample procedure of Experiment 1 was followed except 100 μl of 25 mg·ml⁻¹ IgG (antibody) in 0.9% saline was added and after incubation for 5 minutes, the mixture was added dropwise to 8 g of soybean oil containing 5% w/w Span® 80 under stirring (700 rpm) at 45° C. After 30 minutes of stirring, the water-in-oil (W/O) emulsion was stirred in the ice bath for 20 minutes. The purification of the hydrogel microparticles occurred by washing the w/o emulsion with 1:1 diethyl ether/ethanol mixture (50 ml), followed by centrifugation (˜1000 rpm for 5 minutes). This purification process was repeated twice, followed by purification process with ethanol (1×), and then distilled water (2×). Results are shown in Table 2.

TABLE 2

| Example | Polymer Matrix | Stabilised Photo-thermal | Agent |
|---|---|---|---|
| No | ( % w/w) | particle (% w/w) | ( % w/w) |
| 22 | Agarose hydrogel | P(METAC)-stabilised | IgG (0.25%) |
| | (4%) | AuNPs (0.01%) | |
| 23 | Agarose hydrogel | P(METAC)-stabilised | IgG (0.25%) |
| | (2%) | AuNPs (0.01%) | |
| 24 | Agarose hydrogel | P(METAC)-stabilised | IgG (0.25%) |
| | (4%) | AuNPs (0.05%) | |
| 25 | Agarose hydrogel | P(METAC)-stabilised | IgG (0.25%) |
| | (2%) | AuNPs (0.05%) | |

In order to prepare a formulation for animal injection, the hydrogel microparticles were purified after ethanol washing using sterile water twice, followed by purification using sterile 0.9% saline. After centrifugation, the volume of the microparticles dispersion was adjusted to 2 ml to give a concentration of 0.5 g·ml⁻¹ hydrogel microparticles in aqueous solution.

### Experiment 3

Polymer Composite with Poloxamer Hydrogel Polymer Matrix

A polymer composite comprising IgG, polymer stabilized AuNPs and a poloxamer hydrogel polymer matrix was fabricated similar to those of Examples 11-18 above except with the following differences. Poloxamer (e.g. 200 mg Pluronic® F127/F108 (poloxamer 407 or 338) or 300 mg Pluronic® F87 (poloxamer 237)) was solubilized in 700 mg Milli-Q water at 4° C. 100 μL of 1 mg/mL poly(oligoethylene glycol methyl ether methacrylate)-stabilised AuNPs and 100 μL of 25 mg/mL IgG were sequentially added (at 20° C.). The mixture was rapidly set at 37° C. to solidify the IgG-loaded AuNPs/hydrogel polymer composite for drug release experiment. Results are shown in Table 3.

TABLE 3

| Example | Polymer Matrix | Stabilised Photo-thermal | Agent |
|---|---|---|---|
| No | (% w/w) | particle (% w/w) | (%w/w) |
| 26 | Poloxamer 407 | P(OEGMA)-stabilised | IgG (0.25%) |
| | hydrogel (20%) | AuNPs (0.01%) | |
| 27 | Poloxamer 407 | P(OEGMA)-stabilised | IgG (0.25%) |
| | hydrogel (20%) | AuNPs (0.05%) | |
| 28 | Poloxamer 407 | P(OEGMA)-stabilised | IgG (0.25%) |
| | hydrogel (30%) | AuNPs (0.01%) | |
| 29 | Poloxamer 338 | P(OEGMA)-stabilised | IgG (0.25%) |
| | hydrogel (20%) | AuNPs (0.01%) | |
| 30 | Poloxamer 237 | P(OEGMA)-stabilised | IgG (0.25%) |
| | hydrogel (30%) | AuNPs (0.01%) | |

P(OEGMA) = poly(oligoethylene glycol methyl ether methacrylate).

The ˜1 cm³ polymer composite drug delivery system could be stored as an aqueous solution in the fridge, and prior to drug release experiment the mixture should be vortexed and set at 37° C. again. Due to the thermo-reversible behavior of the poloxamer this polymer composite can be injected directly as a homogenous aqueous solution to the biological environment (physiological temperature of about 37° C.) directly prior to gelation (temperature <30° C.).

### Experiment 4

Fabrication of Microparticles from Polymer Composite with Neat Polymer Matrix

A polymer composite from poly(caprolactone), PCL, was fabricated directly by dissolving polycaprolactone in dichloromethane and mixing with a solution of P(OEGMA)-functionalized AuNPs in an organic solution (e.g. dichloromethane) of hydrophobic drug or water-in-oil (W/O) emulsion of hydrophilic drug/protein. Polymer composites were formed with neat polycaprolactone (PCL) of various molecular weights (2 kDa, 10 kDa and 43 kDa) as the thermoplastic polymer matrix. The polymer composite was fabricated to injectable microparticles, which were prepared by water-in-oil-in-water (W/O/W) double emulsion, followed by solvent aqueous extraction and evaporation. Briefly, bovine serum albumin 50 mg was dissolved in 0.1% (w/v) polyvinyl alcohol (PVA, Mₙ: 30-70 KDa, 90% hydrolyzed) containing phosphate buffer saline (PBS, pH=7.4, 0.1 M) solution as the inner aqueous phase. The inner aqueous phase was emulsified for 20 seconds with methylene chloride solution (oil phase: 15 ml) containing different molecular weight of polycaprolactone (each 500 mg) by sonicator at an output power of 50 W. The resulting first emulsion was then injected at 6 ml/min into a stirred 0.5% (w/v) PVA containing PBS solution (250 ml) as the outer aqueous phase to produce a double W/O/W emulsion. The solution was stirred at 450 rpm in beaker for 30 mins by magnetic stirring PBS solution 200 ml with 0.1% PVA was added and continuously stirred for another 6 h to finish the solvent evaporation at room temperature. The precipitated microspheres were washed by de-ionized water for three times and then freeze-dried overnight and stored at 4° C. before use. The BSA encapsulation efficiency of the PCL microparticles was determined by dissolving the free-dried sample in dichloromethane, followed by multiple extraction in PBS and UV-Vis measurement, which afforded about 45% in average. For drug release experiment, the dry neat polymer composite in the shape of microparticles was dispersed in PBS (pH 7.4) and stored in the fridge (4° C.) until the microparticles were settled prior to measurement. Results are shown in Table 4.

TABLE 4

| Example | Polymer Matrix | Stabilised Photo-thermal | Agent |
|---|---|---|---|
| No | (MW) | particle (% w/w) | (% w/w) |
| 31 | Neat polycaprolactone | P(OEGMA)-stabilised | BSA (4%) |
| | (2 KDa) | AuNPs (0.1%) | |
| 32 | Neat polycaprolactone | P(OEGMA)-stabilised | BSA (4%) |
| | (2 KDa) | AuNPs (0.5%) | |
| 33 | Neat polycaprolactone | P(OEGMA)-stabilised | BSA (4%) |
| | (10 KDa) | AuNPs (0.1%) | |
| 34 | Neat polycaprolactone | P(OEGMA)-stabilised | BSA (4%) |
| | (43 KDa) | AuNPs (0.1%) | |
| 35 | Neat polycaprolactone | P(OEGMA)-stabilised | HRP (4%) |
| | (2 KDa) | AuNPs (0.1%) | |

HRP = horseradish peroxidase

### Experiment 5

Preparation of Microparticle Polymer Composite Encapsulated with Agarose Hydrogel

Polymer composite microparticles with an agent, polymer stabilized AuNPs and 2% agarose were prepared in accordance with the procedure described in Experiment 2. An aqueous dispersion of pre-fabricated and purified agarose-based polymer composite microparticles was mixed (2:1) with 2% low gelling agarose (type VII-A) at 40° C. (e.g 1 ml of 0.5 mg·ml⁻¹ hydrogel microparticle with 0.5 ml of 2% agarose solution). After vortex mixing, the hydrogel microparticles were added dropwise to 8 g of soybean oil containing 2.5% w/w Span® 80 under stirring (700 rpm) at 40° C. After 30 minutes of stirring, the w/o emulsion was stirred in the ice bath for 20 minutes. The purification of the agarose coated polymer composite microparticles occurred by washing the w/o emulsion with 1:1 diethyl ether/ethanol mixture (50 ml), followed by centrifugation (˜1000 rpm for 5 minutes). This purification process was repeated twice, followed by purification process with ethanol (1×), and then distilled water (2×). Results are shown in Table 5.

TABLE 5

| | Polymer | | Stabilised Photo- | |
|---|---|---|---|---|
| Example | Matrix | Coating | thermal particle | Agent |
| No | (% w/w) | (% w/w) | (% w/w) | (% w/w) |
| 36 | Agarose | Low Gelling | P(METAC)- | Bevacizumab |
| | hydrogel | Agarose | stabilised | (0.125%) |
| | (2%) | (1%) | AuNPs | |
| | | | (0.01%) | |
| 37 | Agarose | Low Gelling | P(METAC)- | Bevacizumab |
| | hydrogel | Agarose | stabilised | (0.125%) |
| | (2%) | (1%) | AuNPs | |
| | | | (0.05%) | |

In order to prepare a formulation for animal injection, the polymer coated hydrogel microparticles were purified after ethanol washing using sterile water twice, followed by purification using sterile 0.9% saline. After centrifugation, the volume of the microparticles dispersion was adjusted to 2 ml to give a concentration of 1 mg·ml⁻¹ aqueous solution of hydrogel microparticles coated with 1% agarose.

### Experiment 6

Preparation of Microparticle Polymer Composite Enclosed within Bulk Poloxamer Hydrogel

Polymer composite microparticles with an agent, polymer stabilized AuNPs and 2% agarose as the thermoplastic polymer matrix were prepared in accordance the procedure of Experiment 2. An aqueous dispersion of the pre-fabricated agarose-based hydrogel microparticles were mixed (2:1) with an aqueous solution of 40% Pluronic® F127 (poloxamer 407) at 10° C. (e.g. 1 ml of 0.5 mg·ml⁻¹ agarose-based hydrogel microparticle with 0.5 ml of 40% Pluronic® F127 solution). After vortex mixing, the biphasic mixture was rapidly set at 37° C. to solidify the resultant 20% (w/v) Pluronic® F127 hydrogel that encapsulated the dispersion of agarose-based hydrogel microparticles as an injectable polymer matrix. Results are shown in Table 6.

TABLE 6

| | | Bulk polymer | Stabilised | |
|---|---|---|---|---|
| | Polymer | coating for | Photo-thermal | |
| Example | Matrix | microparticle | particle | Agent |
| No | (% w/w) | (% w/w) | (% w/w) | (% w/w) |
| 38 | Agarose | Poloxamer 407 | P(METAC)- | Bevacizumab |
| | hydrogel | (20%) | stabilised | (0.125%) |
| | (2%) | | AuNPs (0.01%) | |
| 39 | Agarose | Poloxamer 407 | P(METAC)- | Bevacizumab |
| | hydrogel | (20%) | stabilised | (0.125%) |
| | (2%) | | AuNPs (0.05%) | |

The thermoreversible gelation properties of poloxamers means that the coated polymer composite can be stored as an aqueous solution in the fridge, and vortexed for homogenous dispersion of the coated microparticles prior to injection and gelation at physiological temperature (approx. 37° C.) for drug release experiment. The application of a secondary encapsulation or coating to the polymer composite also helps to minimize the premature release of the protein from the surface.

### Experiment 7

Various polymer composites were assessed for softening temperature using both rheometer and by DSC. The results of that comparison are listed in Table 7. Thermal phase transition temperatures provided by DSC measurements can indicate whether a polymer matrix will soften in the composite in the desired temperature range. The softening temperature can be confirmed by rheometer.

TABLE 7

| | | Stabilised | | | Phase |
|---|---|---|---|---|---|
| | | Photo- | | Softening | Transition |
| Exam- | Polymer | thermal | Agent | Temperature | Temperature |
| ple | Matrix | Particle | (% | by | by DSC |
| No | (% w/w) | (% w/w) | w/w) | Rheometer | (Tg or Tin) |
| — | Neat 2 KDa | — | — | 47° C. | 53° C. |
| | poly- | | | | (Tm) |
| | caprolactone | | | | |
| | (100%) | | | | |
| 31 | Neat 2 KDa | P(OEGMA)- | BSA | 43° C. | 46° C. |
| | poly- | stabilised | (4%) | | (Tm) |
| | caprolactone | | | | |
| | (100%) | AuNPs | | | |
| | | (0.1%) | | | |
| — | Neat 10 KDa | — | — | 55° C. | 64° C. |
| | poly- | | | | (Tm) |
| | caprolactone | | | | |
| | (100%) | | | | |
| 33 | Neat 10 KDa | P(OEGMA)- | BSA | 53° C. | 58° C. |
| | poly- | stabilised | (4%) | | (Tm) |
| | caprolactone | AuNPs | | | |
| | (100%) | (0.1%) | | | |
| — | Neat 43 KDa | — | — | 58° C. | 73° C. |
| | poly- | | | | (Tm) |
| | caprolactone | | | | |
| | (100%) | | | | |
| — | Agarose | — | — | 53° C. | 53° C. |
| | Hydrogel | | | | (Tg) |
| | (2%) | | | | |
| 10 | Agarose | P(METAC)- | BSA | 51.5° C. | 52.5° C. |
| | Hydrogel | stabilised | (0.5%) | | (Tg) |
| | (2%) | AuNPs | | | |
| | | (0.01%) | | | |
| — | Agarose | — | — | 56° C. | 57° C. |
| | Hydrogel | | | | (Tg) |
| | (4%) | | | | |
| 11 | Agarose | P(METAC)- | IgG | 54° C. | 54.5° C. |
| | Hydrogel | stabilised | (0.5%) | | (Tg) |
| | (4%) | AuNPs | | | |
| | | (0.01%) | | | |
| — | Poloxamer | — | — | 54° C. | 55.5° C. |
| | 407 | | | | (Tg) |
| | Hydrogel | | | | |
| | (20%) | | | | |
| 26 | Poloxamer | P(OEGMA)- | IgG | 51° C. | 53° C. |
| | 407 | stabilised | (0.25%) | | (Tg) |
| | Hydrogel | AuNPs | | | |
| | (20%) | (0.01%) | | | |
| — | Poloxamer | — | — | 54° C. | 53.5° C. |
| | 237 | | | | (Tg) |
| | Hydrogel | | | | |
| | (30%) | | | | |
| 30 | Poloxamer | P(OEGMA)- | IgG | 50° C. | 49° C. |
| | 237 | stabilised | (0.25%) | | (Tg) |
| | Hydrogel | AuNPs | | | |
| | (30%) | (0.01%) | | | |

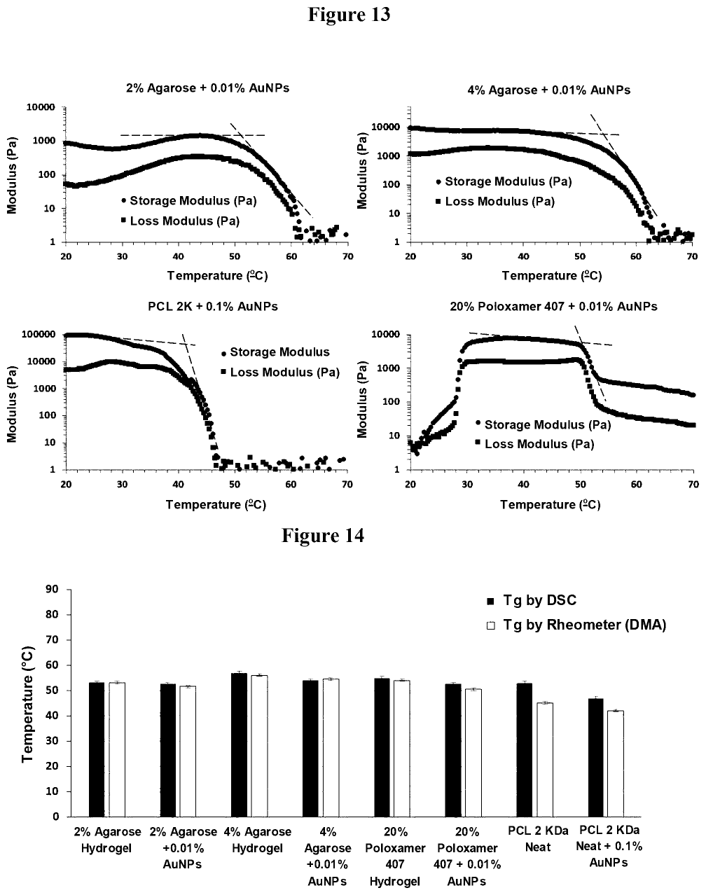

An example of viscosity (storage modulus) change with temperature as measured by rheometry is shown in FIGS. 13 and 14. The softening temperature was determined in accordance with ASTM E1640.

As seen in FIG. 13 the softening point (Tg) of various polymer composites is determined by the intersection of the two tangential lines from the storage modulus according to ASTM E1640. The first tangent line is selected before the transition, while the second tangent line is constructed at the inflection point to approximately the midpoint of the storage modulus drop.

As seen in FIG. 14, the temperature at which the polymer matrix in the composite experiences a thermal phase transition (Tg or Tm) as determined by DSC, correlates well with the softening temperature (Tg) of the polymer matrix as determined by rheometer. The correlations are within ±5° C.

### Experiment 8

Drug Release Experiments

Release of Agent Under Blue Light Exposure

Blue light (400-500 nm) with adjustable power or light intensity was utilized to study the photo-thermal effect of the stabilised AuNPs on the release rate of agents from the polymer composites. It was found that the conversion of photo energy (blue light) to thermal energy by AuNPs when the blue light is turned on (“ON”) led to an increase in the temperature (>44° C.) of the agarose hydrogel polymer matrix, which underwent a reversible phase transition from rigid to viscoelastic hydrogel (decrease in viscosity), thus enhanced the diffusion of the agent from the hydrogel to the PBS solution.

8.1 Release of Lysozyme

An initial release experiment with a polymer composite containing 2.5 mg·ml⁻¹ lysozyme and 0.3 mg·ml⁻¹ P(METAC)-stabilised AuNPs in 4% w/w agarose hydrogel (made a according to Example 4) was conducted. The local temperature increase (ΔT) of the thermoplastic polymer matrix was indicative of the photothermal effect of the polymer-stabilised gold nanoparticles, which heated the agarose polymer matrix to around 50° C., thus increasing the diffusion of the agent (lysozyme). The results of the release experiment are shown in FIG. 2.

As seen in FIG. 2 the release rate of lysozyme (˜14.3 KDa) increased (r_{ON}) with increasing ΔT, when the AuNPs/hydrogel polymer composite was exposed to blue light for about 10 minutes (“ON”). When the blue light was turned off (“OFF”), the temperature of AuNPs/hydrogel polymer composite returned to the initial temperature (˜37° C.), while the release rate of the lysozyme (r_{OFF}) became 5 times lower. This photo-modulated release of lysozyme with r_{ON}>r_{OFF} was observed twice during two ON-OFF cycles under the blue light.

The photo-thermal effect on AuNPs loaded agarose hydrogel was confirmed in the photorheological study, where a ˜22% decrease in shear storage modulus (G′) was observed under the blue light exposure. The temperature-dependant increase in the viscoelasticity of the agarose hydrogel corroborated with increasing release rates of lysozyme at higher temperatures without light.

Other polymer composites prepared in accordance with the Examples were also investigated for their ability to provide for release of the agent in response to light.

8.2 Release of Doxorubicin

It was found that the release rate of a small molecule like doxorubicin (0.2% w/w) from a polymer composite (Example 1) under the exposure to blue light (r_{ON}) was 3 times higher than r_{OFF}.

8.3 Release of Triamcinolone Acetonide

Triamcinolone acetonide (TA) released from a polymer composite (Example 2) exhibited a similar release profile to that of doxorubicin, with 7-12 times higher release rate (r_{ON}) under the exposure to blue light.

8.4 Release of Bovine Serum Albumin (BSA)

A larger molecular weight protein, bovine serum albumin (BSA, ˜66.5 KDa), was photo-modulated released from polymer composites of different agarose content (2% or 4% w/w agarose—Examples 8 and 9). Decreasing the agarose content (from 4% to 2% w/w) did not change the r_{ON} release rate significantly, although the r_{OFF} from the 2% w/w agarose was slightly higher than the r_{OFF} from 4% w/w agarose system, possibly owing to higher diffusion of BSA in the 2% w/w agarose.

8.5 Release of Immunoglobulin G (IgG)

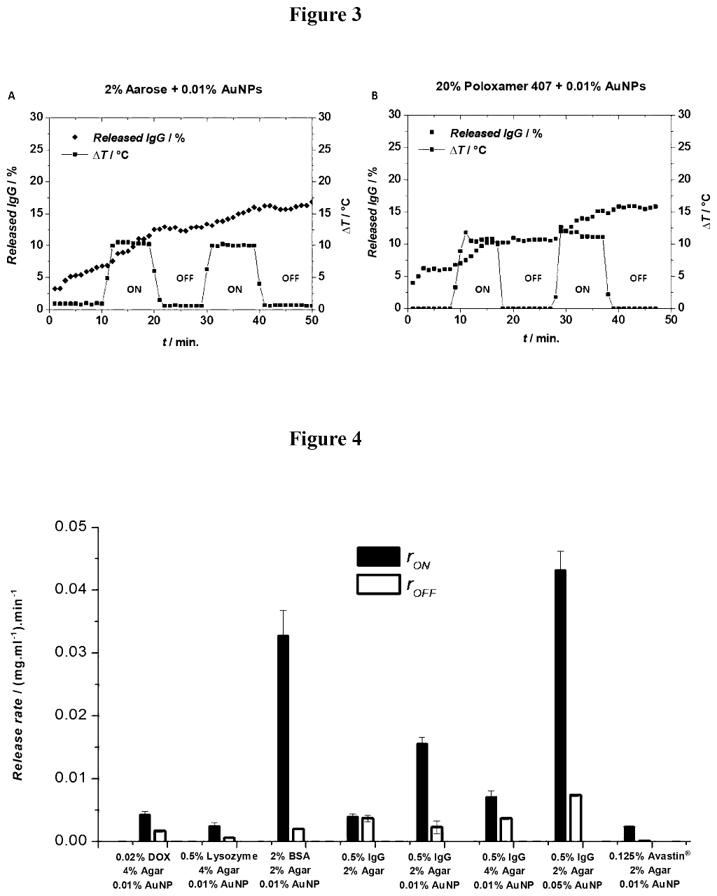

Immunoglobulin G (IgG) (0.5%) was incorporated in polymer composites comprising 0.01% P(METAC)-stabilised AuNPs and agarose hydrogel (2% w/w or 4% w/w—Examples 11 and 12) as the thermoplastic polymer matrix (FIG. 3A). The IgG was then released under the blue light exposure. The incorporation of AuNPs in the agarose hydrogel lead to faster release of IgG (2 times in 4% w/w agarose and 4 times in 2% w/w agarose) under the blue light exposure, compared to a control sample without AuNPs.

Photo-modulated release of IgG (0.25%) was also demonstrated from the thermoplastic polymer matrix comprising 0.01% P(OEGMA)-stabilised AuNPs and poloxamer hydrogel (20-30% w/w) as the thermoplastic polymer matrix. Different types of poloxamer formed an injectable hydrogel at physiological temperature (35-37° C.) above their critical gelation concentration, for example: ≥30% w/w for Pluronic® F87 (poloxamer 237), ≥20% w/w for Pluronic® F127 (poloxamer 407) and F108 (poloxamer 338). The incorporation of stabilised AuNPs in the poloxamer hydrogel lead to faster release of IgG under the blue light exposure, compared to a control sample as following: 10 times in 0.01% AuNPs-loaded 20% w/w poloxamer 407 (Example 26), 40 times in 0.05% AuNPs-loaded 20% w/w poloxamer 407 (Example 27), and 7 times in 0.01% AuNPs-loaded 30% w/w poloxamer (Example 28). The real-time temperature measurement of the 0.01% AuNPs- and 0.05% AuNPs-loaded 20% w/w poloxamer 407 hydrogel revealed under the light exposure local temperature increases up to 50° C. and 55° C., respectively, in comparison with the surrounding environment (PBS medium at 37° C.), which corroborated with the increase of their r_{ON} release rates (FIG. 3B).

8.6 Release of Bevacizumab (Avastin®)

Bevacizumab (Avastin®) was released from the polymer composite of Example 19 (2% w/w agarose+0.1 mg·ml⁻¹ AuNPs and Avastin® 0.125% or 1.25 mg·ml⁻¹) under blue light. The presence of AuNPs at concentrations of 0.01% and 0.05% resulted in 2 times and 17 times higher release rates respectively, under the blue light exposure, compared to a control sample without AuNPs.

The release rates (r_{ON} and r_{OFF}) of different agents released from the agarose hydrogel upon blue light exposure are summarized in FIG. 4.

### Experiment 9

Effect of Stabilized Photo-Thermal Particle Concentration and Light Intensity on Release of Agents

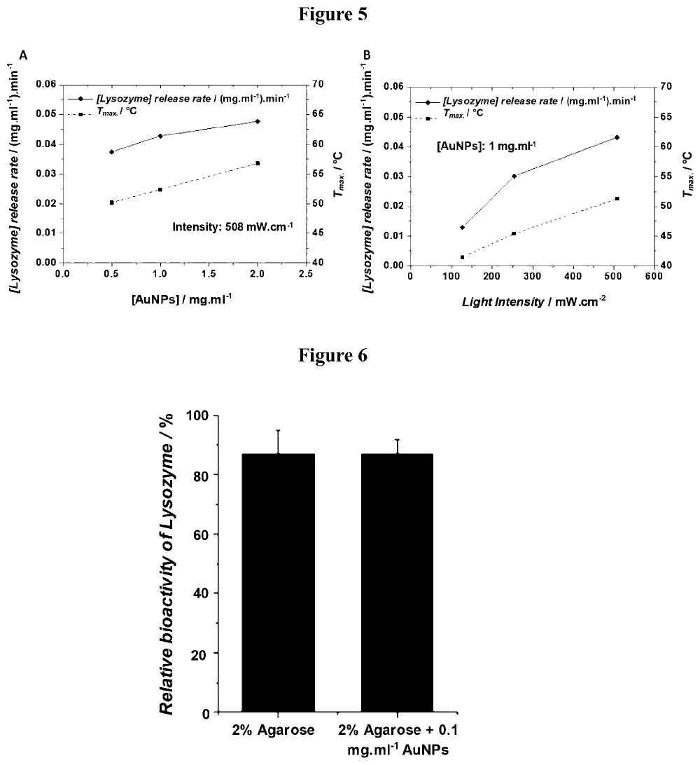

The photo-thermal effect of stabilised AuNPs was investigated by exposing polymer composites with different concentration of stabilised AuNPs (Examples 5 to 7) to light at a constant intensity (˜508 mW·cm⁻²). The maximum local temperature of polymer composites with increasing amounts of AuNPs increased under the blue light exposure. The release rate of lysozyme also increased slightly (FIG. 5A).

In polymer composites loaded with IgG and comprising either 2% or 4% agarose hydrogel as the thermoplastic polymer matrix, an increase in the stabilised AuNPs concentration from 0.01% to 0.05% led to an increase in the release rate (r_{ON, 1st}) of the IgG (5 times in 4% w/w agarose and 11 times in 2% w/w agarose).

The effect of the blue light on the release of lysozyme from a polymer composite (Example 6) was investigated by varying the blue light intensity (127, 254, and 508 mW·cm⁻²). At constant AuNPs concentration (1 mg·ml⁻¹ AuNPs), the maximum local temperature of polymer composite and the release rate of lysozyme increased with higher light intensity, demonstrating the effect of light intensity on the release of lysozyme (FIG. 5B).

Through a combination of varying AuNPs concentration and/or light intensity, the release rate of each agent can be tuned.

### Experiment 10

Effect of Photoenergy on the Bioactivity of Released Agent

The bioactivities of some of the proteins released from a polymer composite of the invention as well as a comparative polymer composite without photo-thermal particles were tested according to the procedure described above.

10.1 Lysozyme

Lysozyme released from a polymer composite containing 2% w/w agarose with 0.1 mg·ml⁻¹ AuNPs (Example 3) exhibited relative bioactivity above 85%, even after blue light exposure. Lysozyme released from a comparative polymer composite containing 2% w/w agarose but without AuNPs exhibited a similar relative bioactivity of 85% (FIG. 6). The result demonstrates that the photo-thermal particles and the heat generated by the particles do not have an adverse effect on the bioactivity of the released agent.

10.2 Bevacizumab/Avastin®

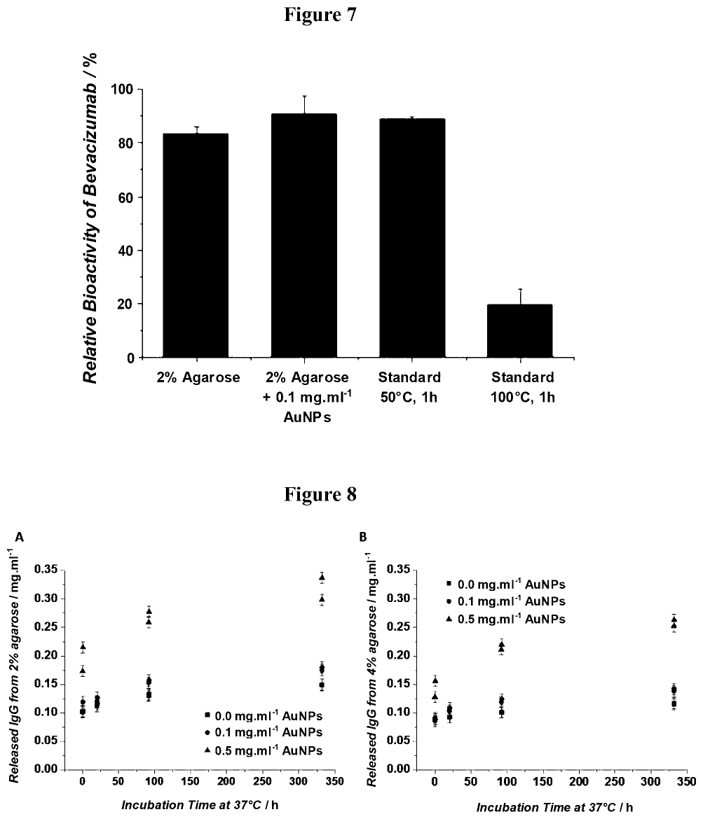

Bevacizumab (Avastin®) released from the polymer composite of Example 19 (2% agarose, with and without 0.1 mg·ml⁻¹ [AuNPs]) were assessed for bioactivity after three ON-OFF cycles (total 70 mins) in accordance with the procedure described above. The samples of released Avastin® were isolated for quantification and bioactivity test using ELISA. This ELISA sandwich assay was clinically developed using specific affinity of Avastin® to human VEGF-165 (165 isoforms of VEGF) in the anti-angiogenesis therapy. The concentration of biologically active Avastin® (VEGF-165 binding Avastin®) was in linear colleration with the fluorescence intensity of ABTS substrate at 405 nm, which was oxidized by the Avastin®-binding horseradish peroxidase conjugate of IgG (in ng·ml⁻¹). A comparison of released Avastin® concentration determined by ELISA method and UV-Vis calibration resulted in the estimated bioactivity of Avastin® at 83-87% in regards to its VEGF-165 binding activity. As negative control, an Avastin® standard (10 ng·ml⁻¹) was heated up to 50° C. and 100° C. for 1 hour, the supernatant was subjected to ELISA after centrifugation. The denatured antibody (100° C.) resulted in 19% bioactivity, while heating at 50° C. or the photothermal effect from the AuNPs and blue light did not significantly damage the functional structure and bioactivity of the Avastin®. The results are shown in FIG. 7. The measurements were carried out in triplicate, while the error bars represent the standard deviation.

### Experiment 11

Injectability of Polymer Composite with Hydrogel Polymer Matrix Fabricated as Microparticles

Polymer composite microparticles containing 2% or 4% agarose hydrogel polymer matrix, polymer-stabilised AuNPs (0.0%, 0.01% and 0.05% w/w) and IgG antibody (0.25%) were prepared in accordance with the procedure described above, through the emulsion modification of the bulk hydrogel samples (Examples 22 to 25).

The hydrogel microparticles possessed good colloidal stability and can be transferred and injected easily by syringe with 30-gauge needle. Optical microscopy showed the size of the spherical microparticles below 50 μm.

### Experiment 12—Long Term Release Studies

12.1 IgG

The long term release of IgG from the hydrogel microparticles was also investigated in accordance with the procedure described above. To carry out the study, the microparticles were sedimented in a PBS buffer, while the concentration of released agent was measured in the supernatant before and after the exposure to blue light. Different concentrations of AuNPs and agarose concentration were used to study the effect of these parameters on the photo-responsiveness and the agent release profile of the microparticles. In every measurement hydrogel microparticles were exposed to blue light (˜500 mW·cm⁻²) for 10 minutes, and the concentrations of released IgG before and after exposure were plotted over period of time. The results are shown in FIG. 8.

As seen in FIG. 8, higher AuNPs loading (0.5 and 0.1 mg·ml⁻¹ AuNPs) lead to higher response of the microparticles to blue light, which is represented through a significant increase of agent concentration before and after the exposure to light. Microparticles without AuNPs (0.0 mg·ml⁻¹) did not show any significant increase before and after the light exposure. Lower agarose content increased slightly the release rate of the agent, and increased the response of the microparticles to blue light. This study confirmed the photo-thermal effect of AuNPs in the microparticles through the combination of AuNPs and agarose concentrations, which can be customized to obtain desired release profile for specific agent.

The structural integrity of the released bevacizumab from the agarose hydrogel bulk as well as the hydrogel microparticles were assessed using gel filtration chromatography to provide higher than 90% retained monomeric full antibody structure (<10% degradation and aggregation products).

12.2 Albumin (Using Polymer Mixtures)

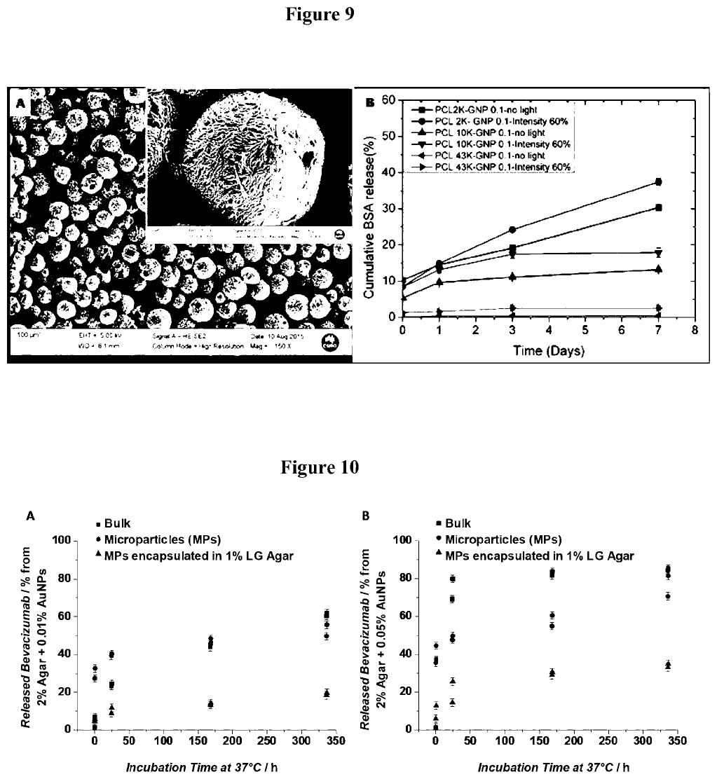

Polymer composite microparticles with neat PCL polymer matrix were also prepared in accordance with the procedure described above. Due to their thermoplastic property, PCL was considered as an appropriate polymer matrix. The advantage of PCL is its biodegradation and hydrophobicity to encapsulate and release hydrophobic drugs. In order to design the PCL as a polymer matrix to deliver hydrophilic drugs or protein, modification of PCL using emulsion was necessary to produce a porous PCL film. Using double emulsion technique, this porous PCL matrix can be modified further to porous microparticles that contain gold nanoparticles for photo-modulated delivery of hydrophilic drug/protein. For example, PCL with different molecular weight (2 KDa, 10 KDa, and 43 KDa) were modified in the W/O/W double emulsion, and blended with P(OEGMA)-stabilised AuNPs (0.1-0.5%) and BSA (˜4% w/w) aqueous solution stabilized with PVA (0.5% w/w). The resultant PCL microparticles (average diameter 40±10 μm by SEM) with the porosity (<4 μm by SEM) were obtained as dry sample (FIG. 9A). In the drug release experiment for 7 days incubation at 37° C. in PBS solution (4 times of 10 mins photo exposure), the release rate of BSA from 0.1% AuNPs-loaded 2 KDa PCL microparticles was 1.3 times higher under the exposure of blue light, compared with the control sample (without light). This on-off release ratio decreased with increasing PCL molecular weight (Examples 31 to 34). In comparison with the 10 KDa and 43 KDa PCL microparticles containing the same 0.1% AuNPs, the 2 KDa PCL microparticles released 2 times and 10 times more BSA over the period of 7 days, respectively (FIG. 9B). Structural integrity test of the released BSA using circular dichroism proved the stability of the protein after its photo-modulated release. The enzymatic activity of the released horseradish peroxidase was about 73% retained after its photo-modulated release from the 2 KDa PCL microparticles.

### Experiment 13—Polymer Composite Coated with Additional Polymer

13.1 Polymer Composite Microparticles Coated by Polymer

Hydrogel-based polymer composite microparticles coated with another hydrogel layer were also prepared in accordance with the procedures described above.

A long-term release study of bevacizumab from a polymer composite microparticle surface coated with 1% w/w low gelling (LG) agarose (Examples 36 and 37) displayed a decreased release rate for the bevacizumab, showing that the additional LG agarose coating can help to minimize the burst release of bevacizumab from the hydrogel microparticles (FIG. 10). In order to increase the response of this coated microparticles to blue light, AuNPs (0.1-0.5 mg·ml⁻¹) can also be incorporated in the polymer coating.

The release rate of a drug can be adjusted by adjusting the agarose content of the hydrogel microparticle, AuNPs loading, and coating, in order to meet the pharmacokinetic and pharmacodynamic of the drug.

13.2 Polymer Composite Microparticles Coated by Bulk Polymer

Pre-fabricated polymer composite microparticles with agarose hydrogel polymer matrix were mixed with, an aqueous solution containing 20% (w/w) of poloxamer 407 (Examples 38 and 39). The resulting microparticle/poloxamer 407 solution formed a hydrogel once injected to a biological environment at 35-37° C. The bulk poloxamer encapsulated the polymer composite microparticles.

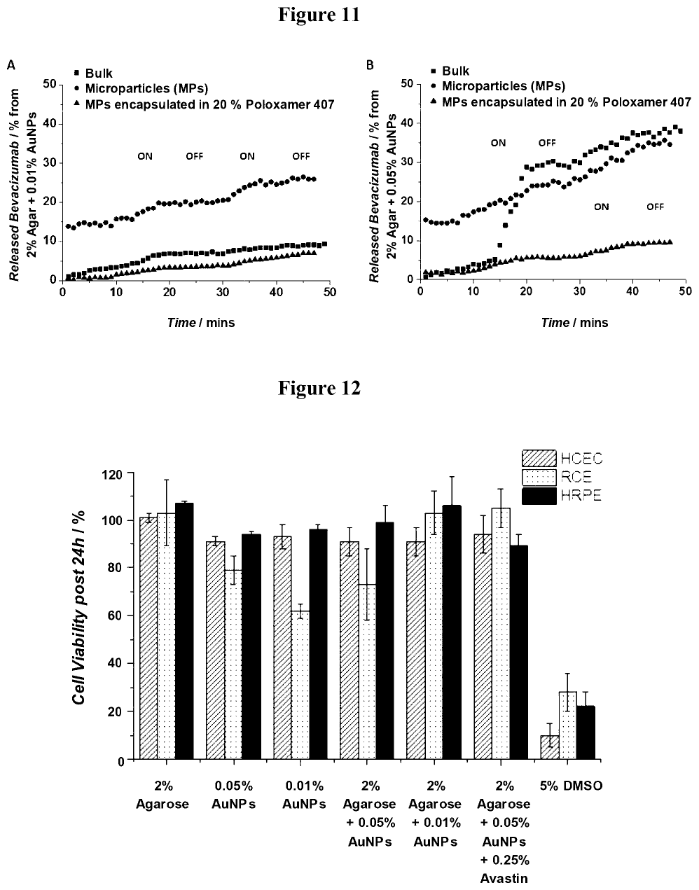

When the coated polymer composite microparticles were exposed to light, it was found that burst release of the agents directly from the high surface area of the agarose microparticles were minimized by the poloxamer hydrogel coating, as demonstrated in the real-time photo-modulated release experiment. The results are shown in FIG. 11. Polymer-stabilized AuNPs can be incorporated to this bulk poloxamer hydrogel as well to increase the on-off release rate ratio of the drug delivery system.

### Experiment 14

In Vitro Toxicity of Polymer Composite Microparticles on Ocular Cells

The in vitro toxicity of hydrogel microparticles to ocular cells was assessed by incubating human retinal pigment epithelial cells (HRPE or ARPE-19), human corneal epithelial cells (HCE), and rabbit corneal endothelial cells (RCE) with hydrogel microparticles containing 0.5 mg·ml⁻¹ AuNPs and 4% agarose in accordance with the procedure described above (Example 24). This sample was diluted to study the concentration dependant toxicity to give 0.25 and 0.125 mg·ml⁻¹ AuNPs. Comparative samples of hydrogel microparticles without AuNPs (4% agarose only) and AuNPs aqueous solution (same concentration of gold nanoparticles as in the AuNPs-loaded hydrogel microparticles) were used as control samples.

After 24 h and 48 h incubation, all ocular cells exhibited cell viability above 70%, which indicated that the hydrogel microparticles did not induce any cytotoxicity. The results are shown in FIG. 12.

### Experiment 15

Preliminary In Vivo Animal Safety Study of Injected Polymer Composite Hydrogel Microparticles

A solution containing 0.5 mg/ml of polymer composite with hydrogel microparticles (comprising 0.05% AuNPs, 0.25% bevacizumab 4% w/w agarose) and comparative solutions containing 0.5 mg/ml of AuNPs only and 0.5 mg/ml of 4% agarose microparticles only (no AuNPs) were prepared in accordance with the procedure described above (Example 24). 100 μL of each sample were injected via syringe with a 30-gauge needle to the right subconjunctival of the rabbits for the preliminary in vivo study. The contralateral eye (left eye) was used as a control.

In the preliminary in vivo study, it was observed that the polymer composite were small enough to pass the 30-gauge needle. Purple area of injection was observed, indicating the implantation of the polymer composite hydrogel microparticles. The injected eyes of two rabbits were exposed to blue light for 2 minutes. Most of the rabbits survived the anaesthesia, and were alive for more than one week after the injection. In order to study the biocompatibility of the injected materials, slit-lamp microscopy of all the rabbits were performed after 2 h, 1 day, and 3 days of the injection. No abnormalities on the anterior segment or cornea of the eye were observed. No infection was observed as well, indicating the sterile condition of the samples and injection. After 1 day of injection hyperaemia near the injection site was observed. This could be caused by the materials or the injection itself, which has to be compared with an injection of saline.

The effect of injected polymer composite on the posterior segment was investigated using electroretinography (ERG). The retina responses of the rabbits to standardized light stimuli were measured using flashes at attenuated/amplified intensity to give typical curves containing the a-wave (initial negative deflection) and the b-wave (positive deflection). The signals from the injected eye (right eye) and the control/contralateral eye (left eye) were measured at the same time for comparison. A signal reduction in a-wave, b-wave, and 20 Hz amplitude from the injected eye could indicate a negative effect or toxicity on the retinal photoreceptors of the injected eye.

A rabbit that was injected with hydrogel microparticles only (no AuNPs) did not exhibit any retinal toxicity. One of three rabbits that were injected with polymer composite hydrogel microparticles exhibited a slight reduction of signal in the injected eye. Meanwhile, the other two rabbits injected with polymer composite did not exhibit any retinal toxicity, although they were injected with the same sample and exposed to blue light. When pulse ERG was performed, we did not observe significant reduction of 20 Hz amplitude. This confirmed that the injected samples did not induce toxicity to the photoreceptors on the retina of the rabbits.

As shown above, a facile fabrication method of agarose-based hydrogel loaded with polymer-stabilised gold nanoparticles (AuNPs) and an agent such as a drug or protein, for photo-modulated drug delivery applications has been established. Different agent from small molecules (doxorubicin, triamcinolone acetonide) to protein (lysozyme, bovine serum albumin) and antibody (immunoglobulin G, Avastin®) have exhibited sustained released from the AuNPs-loaded hydrogel polymer composite, as well as released at higher rate under the exposure of blue light. Several parameters, such as agarose content, concentration of AuNPs, and blue light intensity, have been investigated to adjust the release profile of the agent, depending on the type and size of the agent. Released lysozyme and Avastin® from the polymer composite hydrogel exhibited above 80% biological activities after blue light exposure, in particular Avastin® due to its VEGF binding activity in the anti-angiogenesis therapy. This polymer composite can be modified to microparticles for injection via 30-gauge needle. The release rate of agent from these polymer composite modified into hydrogel microparticles was generally higher due to higher surface area. Coating of the microparticles using agarose can be applied to reduce the release rate of agent. These polymer composite hydrogel microparticles exhibited low cytotoxicity against cancer cells and ocular cells. In the preliminary in vivo study, these polymer composite hydrogel microparticles can be injected into the eye of a rabbit, and did not induce any corneal and retinal abnormalities. Due to its biocompatibility and versatility, the photo-modulated polymer composites have potential for ophthalmic applications, such as ‘on-demand’ light-controllable injection of therapeutics for the treatment of ocular diseases.

### Experiment 16

Increasing the Storage Modulus of the Poloxamer-Based Composite Using Covalent Cross-Linking

In order to increase the inherent storage modulus of 18.5% (w/w) poloxamer 407 hydrogel, an additional covalent cross-linking process was introduced. The addition of photo-curable acrylate groups to the poloxamer was able to increase its storage modulus upon photo-catalytically cross-linking process. By increasing the storage modulus of the self-assembled poloxamer matrix, the stability of the polymer in the aqueous environment can be greatly improved. The introduction of a cross-linking agent, poloxamer 407 diacrylate, can be prepared by the modification of poloxamer 407 using acryloyl chloride and triethyl amine following the method from the literature (Di Biase et al, Soft Matter 2011, 7, 4928-4937).

A 1:3 mixture of poloxamer 407 diacrylate and native poloxamer 407 (total concentration 18.5% w/w) was dissolved in PBS (pH 7.4) containing 0.1% FITC-BSA, 0.1% Irgacure 2959 and P(OEGMA)-functionalized AuNPs. Upon the inherent gelation process (self-assembly) at 37° C., the pre-formed polymer composite was exposed to UV (365 nm, 80 mWcm⁻², 600 s). In comparison with 18.5% (w/w) pure native poloxamer 407 (no diacrylate), about 67% increase of storage modulus was observed in the oscillatory shear rheometer. The results are shown in Table 8.

TABLE 8

| Example | Polymer Matrix | Stabilised Photo-thermal | Agent |
|---|---|---|---|
| No | (% w/w) | particle (% w/w) | (% w/w) |
| 31 | Poloxamer 407 | P(OEGMA)-stabilised | FITC-BSA |
| | hydrogel (18.5%) | AuNPs (0.01%) | (0.1%) |
| 32 | 1:3 poloxamer 407 | P(OEGMA)-stabilised | FITC-BSA |
| | diacrylate/poloxamer | AuNPs (0.01%) | (0.1%) |
| | 407 hydrogel (18.5%) | | |

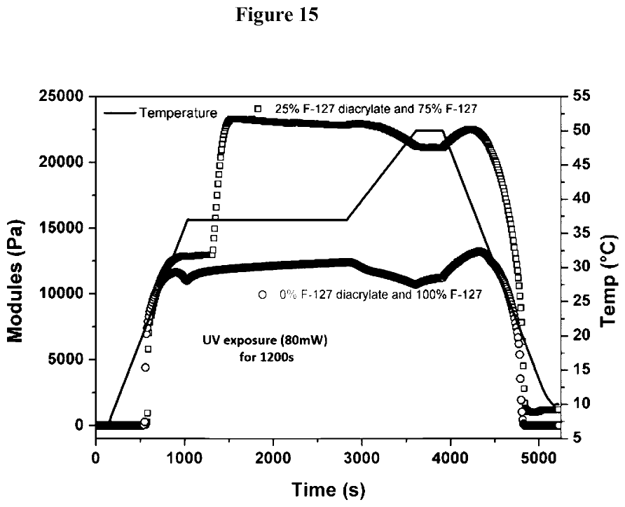

The softening point of the polymer composites was assessed by rheometer. It was found that softening point shifted from ˜39° C. to ˜45° C. through the addition of acrylate groups and photo-curing process, when the temperature of the sample was increased. The storage modulus of the polymer composite can therefore be increased, while the softening point at elevated temperature can be shifted. The results are shown in FIG. 15.

It is to be understood that various other modifications and/or alterations may be made without departing from the spirit of the present invention as outlined herein.

1. A polymer composite for controlled release of an agent comprising:
a thermoplastic polymer matrix;
an agent dispersed within the thermoplastic polymer matrix; and
a plurality of photo-thermal particles dispersed in the thermoplastic polymer matrix, the particles having a non-carboxylic acid stabiliser bound thereto;
wherein the thermoplastic polymer matrix has a softening point when in the composite in the temperature range of from about 30° C. to 70° C. as determined by rheometer, and wherein upon exposure of the composite to photo energy, the particles absorb the photo energy and emit thermal energy to promote the softening of the thermoplastic polymer matrix and modulate the release of the agent from the thermoplastic polymer matrix.

2. A polymer composite according to claim 1, wherein the non-carboxylic acid stabiliser comprises a neutral or charged polymer.

3. A polymer composite according to claim 1, wherein the non-carboxylic acid stabiliser comprises a cationic polymer.

4. (canceled)

5. A polymer composite according to claim 1, wherein the non-carboxylic acid stabiliser is covalently bound to the photo-thermal particles via a functional group selected from the group consisting of a thiol, thiocarbonylthio and amino functional group.

6. (canceled)

7. A polymer composite according to claim 1, wherein the thermoplastic polymer matrix is in the form of a hydrogel comprising a polymer phase and an aqueous liquid phase.

8. A polymer composite according to claim 7, wherein the polymer phase of the hydrogel comprises a polymer selected from the group consisting of a polysaccharide, a polypeptide, a polyether, a polyester, a poly(vinyl alcohol), a poly(vinyl pyrrolidone), poly(ethylene-vinyl acetate) and a poloxamer.

9. A polymer composite according to claim 6, wherein the polymer phase of the hydrogel comprises a polysaccharide selected from the group consisting of agarose, carrageenan, chitosan, gellan gum, starch, alginate, hyaluronic acid, dextran, cellulose and mixtures thereof.

10. (canceled)

11. (canceled)

12. (canceled)

13. A polymer composite according to claim 1, wherein the thermoplastic polymer matrix is in the form of a neat polymer film.

14. A polymer composite according to claim 13, wherein the thermoplastic polymer matrix comprises at least one neat polymer selected from a polyester, a polyamide, a polyoxazoline, a polyether and poly(vinyl pyrrolidone).

15. (canceled)

16. (canceled)

17. A polymer composite according to claim 13, wherein the thermoplastic polymer matrix comprises neat polycaprolactone.

18. A polymer composite according to claim 17, wherein the polycaprolactone has a molecular weight (Mn) in a range of from about 1000 g/mol to 43,000 g/mol.

19. A polymer composite according to claim 1, wherein the stabilised photo-thermal particles absorb photo-energy having a wavelength in a range selected from the group consisting of from about 10 nm to about 1 mm, from about 365 nm to about 1400 nm, and from about 400 nm to about 900 nm.

20. (canceled)

21. A polymer composite according to claim 1, wherein the photo-thermal particles are gold particles.

22. A polymer composite according to claim 21, wherein the gold particles have a diameter in a range selected from the group consisting of from about 5-400 nm, from about 10-200 nm, from about 20-100 nm, and from about 40-80 nm.

23. A polymer composite according to claim 21, wherein the gold particles are present in the polymer composite in an amount of from about 0.01 to 10.0 mg/ml or 0.001 to 1.0% (w/v).

24. A polymer composite according to claim 1, wherein the agent is a drug.

25. (canceled)

26. A polymer composite according to claim 1, wherein the polymer composite is in the shape of spherical particles with a size comprised between 1 μm and 1000 μm or between 10 μm and 200 μm.

27. A process for the preparation of a polymer composite of claim 1, the process comprising the steps of forming a liquid polymer mixture comprising at least one polymer, at least one agent and a plurality of non-carboxylic acid stabilised photo-thermal particles, and solidifying the liquid polymer mixture to form the polymer composite.

28. (canceled)

29. (canceled)

30. (canceled)

31. (canceled)

32. An implantable article comprising a polymer composite of claim 1, and a thermoplastic polymer coating or enclosing the polymer composite.

33. An ocular implant comprising a polymer composite according to claim 1.
